(19) United States
(12) Patent Application Publication
(10) Pub. No.: US 2024/0150580 A1
(43) Pub. Date: May 9, 2024

(54) INTERPENETRATING POLYMER NETWORKS FOR USE IN SUTURABLE AND FLEXIBLE MATERIALS

(71) Applicant: Lung Biotechnology PBC, Silver Spring, MD (US)

(72) Inventors: Aman Kaur, Manchester, NH (US); Lina Trigg, Mason, OH (US); Barbara Nsiah, Manchester, NH (US); Luis Alvarez, Lexington, MA (US)

(73) Assignee: Lung Biotechnology PBC, Silver Spring, MD (US)

(21) Appl. No.: 18/226,635

(22) Filed: Jul. 26, 2023

Related U.S. Application Data
(60) Provisional application No. 63/392,250, filed on Jul. 26, 2022.

(51) Int. Cl. C09D 4/06; B33Y 70/00; C12N 5/071; C12N 11/087

(52) U.S. Cl. CPC C09D 4/06; B33Y 70/00; C12N 5/0688; C12N 11/087; B33Y 10/00

(57) ABSTRACT

An advanced manufactured interpenetrating polymer network (AM-IPN) comprising: a primary polymer network; a secondary polymer network, wherein the secondary polymer network is bonded to the primary polymer network via one or more crosslinks, wherein one or more of the primary polymer network, the secondary polymer network and the one or more crosslinks are printed using a synthetic bioink is disclosed. Methods of making and using are also disclosed.

### CROSS-REFERENCE TO RELATED APPLICATIONS

This application claims priority to U.S. Provisional Application No. 63/392,250, filed Jul. 26, 2022, which is incorporated by reference in its entirety.

### TECHNICAL FIELD

The present application relates to 3D printed structures, namely, 3D printed interpenetrating polymer networks (IPN) for use in suturable and flexible hydrogel materials.

### BACKGROUND

Interpenetrating polymer networks (IPN) permit development of hydrogel materials that may be sutured and manipulated (e.g., bent, twisted, flexed) without fracture and that may be used for perfusion and ventilation.

There is a need for IPNs may be 3D printed as suturable and flexible hydrogel materials for organ additive manufacturing.

### SUMMARY

The systems and methods of the present disclosure include 3D printed interpenetrating polymer networks (IPN) for use in suturable and flexible hydrogel materials using synthetic bioinks.

In an embodiment, an advanced manufactured interpenetrating polymer network (AM-IPN) comprises: a primary polymer network; a secondary polymer network, wherein the secondary polymer network is bonded to the primary polymer network via one or more crosslinks. In an embodiment, the one or more of the primary polymer network, the secondary polymer network and the one or more crosslinks are printed using a synthetic bioink.

In an embodiment, the synthetic bioink comprises one or more of: HPA, from about 5% to about 20%, and any range or value there between; PEGDA 6000, from about 5% to about 20%, and any range or value there between; TMPTA 912, from about 0.5% to about 3%, and any range or value there between; NAP, from about 0.5% to about 3%, and any range or value there between; UV386A, from about 0.1% to about 0.5%, and any range or value there between; Polymer, from about 0.001% to about 2.0%, and any range or value there between; and Water, in an amount as a balance.

In an embodiment, the polymer comprises one or more of: polyethylene oxide (PEO), polyethylenimine (PEI), polyvinylpyrrolidone (PVP), polyvinyl alcohol (PVA), and combinations thereof.

In an embodiment, the polymer is PEO having a molecular weight from about 100000 to about 4000000, and any range or value there between.

In an embodiment, the polymer is PEO in an amount from about 0.005% to about 1%, and any range or value there between.

In an embodiment, the polymer is PEI having a molecular weight from about 25000 to about 75000, and any range or value there between.

In an embodiment, the polymer is PEI in an amount from about 0.005% to about 1%, and any range or value there between.

In an embodiment, the polymer is PVP having a molecular weight from about 1000000 to about 1300000, and any range or value there between.

In an embodiment, the polymer is PVP in an amount from about 0.005% to about 2%, and any range or value there between.

In an embodiment, the polymer is PVA having a molecular weight from about 89000 to about 98000, and any range or value there between.

In an embodiment, the polymer is PVA in an amount from about 0.005% to about 2%, and any range or value there between.

In an embodiment, the synthetic bioink comprises one or more of: HPA, in an amount from about 5% to about 20%, and any range or value there between; PEGDA 3400, in an amount from about 5% to about 20%, and any range or value there between; NAP, in an amount from about 0.5% to about 3%; UV386A, in an amount from about 0.1% to about 0.5%, and any range or value there between; Polymer, in an amount from about 0.001% to about 2.0%, and any range or value there between; and Water, in an amount as a balance.

In an embodiment, the polymer comprises one or more of: polyethylene oxide (PEO), polyethylenimine (PEI), polyvinylpyrrolidone (PVP), polyvinyl alcohol (PVA), and combinations thereof.

In an embodiment, the polymer is PEO having a molecular weight from about 100000 to about 4000000, and any range or value there between.

In an embodiment, the polymer is PEO in an amount from about 0.005% to about 1%, and any range or value there between.

In an embodiment, the polymer is PEI having a molecular weight from about 25000 to about 75000, and any range or value there between.

In an embodiment, the polymer is PEI in an amount from about 0.005% to about 1%, and any range or value there between.

In an embodiment, the polymer is PVP having a molecular weight from about 1000000 to about 1300000, and any range or value there between.

In an embodiment, the polymer is PVP in an amount from about 0.005% to about 2%, and any range or value there between.

In an embodiment, the polymer is PVA having a molecular weight from about 89000 to about 98000, and any range or value there between.

In an embodiment, the polymer is PVA in an amount from about 0.005% to about 2%, and any range or value there between.

In an embodiment, a method of making an advanced manufactured interpenetrating polymer network (AM-IPN) comprises: printing one or more of a primary polymer network, a secondary polymer network and one or more crosslinks using a synthetic bioink and a 3D printing technique; and assembling and/or printing the AM-IPN to form an assembled AM-IPN.

In an embodiment, the synthetic bioink comprises one or more of polyethylene oxide (PEO), polyethylenimine (PEI), polyvinylpyrrolidone (PVP), polyvinyl alcohol (PVA), and combinations thereof.

In an embodiment, the AM-IPN is printed at from about 50 mW/cm² to about 400 mW/cm², and any range or value there between.

In an embodiment, the synthetic bioink comprises polyethylene oxide (PEO) in an amount from about 0.005% to about 1%, and any range or value there between, and the AM-IPN is printed at 50 mW/cm² to about 300 mW/cm², any range or value there between.

In an embodiment, the synthetic bioink comprises polyethylenimine (PEI) in an amount from about 0.005% to about 1%, and any range or value there between, and the AM-IPN is printed at from about 150 mW/cm² to about 300 mW/cm², and any range or value there between

In an embodiment, the synthetic bioink comprises polyvinylpyrrolidone (PVP) in an amount from about 0.005% to about 2%, and any range or value there between, and the AM-IPN is printed at from about 50 mW/cm² to about 300 mW/cm², and any range or value there between.

In an embodiment, the synthetic bioink comprises polyvinyl alcohol (PVA) in an amount from about 0.005% to about 2%, and any range or value there between, and the AM-IPN is printed at from about 100 mW/cm² to about 300 mW/cm², and any range or value there between.

In an embodiment, the 3D printing technique is one or more of digital light projection printing (DLP), stereolithography (SLA) printing technique, extrusion 3D printing technique or selective laser sintering 3D printing technique or a combination thereof.

In an embodiment, the 3D printing technique is a digital light printing (DLP) printing technique.

In an embodiment, a method of using an advanced manufactured interpenetrating polymer network (AM-IPN) comprises: modifying a surface of the AM-IPN to attach small airway epithelial cells (SAEC) to form a modified AM-IPN. In an embodiment, the method further comprises: using the modified AM-IPM for perfusion and ventilation.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of a required fee.

The details of one or more implementations of the subject matter described in this specification are set forth in the accompanying drawings and the description below. Other features, aspects, and advantages of the subject matter will become apparent from the description, the drawings, and the claims.

FIG. 1A illustrates an interpenetrating polymer network in a synthetic AA42 Bioink with 8% PEGDA 3400, showing a high crosslinking density;

FIG. 1B illustrates an interpenetrating polymer network in a synthetic AI28 Bioink with 5% PEGDA 6000 and 3% Triactetate, showing a moderate crosslinking density;

FIG. 1C illustrates an interpenetrating polymer network in a synthetic 602 Bioink with 5% PEGDA 6000, showing a low crosslinking density;

FIG. 2A illustrates a photograph of an air supply for a TA instrument with an OmniCure light source and a TRIOS software package;

FIG. 2B illustrates a photograph of the OmniCure light source for the TA instrument of FIG. 2A.

FIG. 2C-1 illustrates a first screen shot of the TRIOS software package for the TA instrument of FIGS. 2A-2B, showing a geometry icon under an experiment tab.

FIG. 2C-2 illustrates a second screen shot of the TRIOS software package for the TA instrument of FIGS. 2A-2B, showing an instrument tab;

FIG. 2C-3 illustrates a third screen shot of the TRIOS software package for the TA instrument of FIG. 2A, showing inertia (calibration) and oscillatory (new mapping) settings under the instrument tab;

FIG. 2C-4 illustrates a fourth screen shot of the TRIOS software package for the TA instrument of FIGS. 2A-2B, showing accessories setting under a Calibration tab;

FIG. 2C-5 illustrates a fifth screen shot of the TRIOS software package for the TA instrument of FIGS. 2A-2B, showing a Geometries tab;

FIG. 2C-6 illustrates a sixth screen shot of the TRIOS software package of the TA instrument of FIGS. 2A-2B, showing condition options and settings;

FIG. 2C-7 illustrates a seventh screen shot of the TRIOS software package of the TA instrument of FIGS. 2A-2B, showing other event UV settings;

FIG. 2C-8 illustrates an eighth screen shot of the TRIOS software package of the TA instrument of FIGS. 2A-2B, showing oscillation fast sampling settings;

FIG. 2D illustrates a graph of Step Time ts (s) vs. Storage Modulus G′ (kPa) of an advanced manufactured interpenetrating polymer network (AM-IPN) for Polyethylene Oxide (PEO) in synthetic AL35, AL36, AL37 and AL38 Bioinks;

FIG. 3A illustrates an air supply for a DMA 850 instrument with a TRIOS software package. The air supply should be about 60 psi to the DMA instrument;

FIG. 3B illustrates a photograph of calibrating a bottom clamp for the DMA 850 instrument of FIG. 3A;

FIG. 3C illustrates a first screen shot of the TRIOS software package for the DMA 850 instrument of FIGS. 3A-3B, showing a Clamp Calibration tab;

FIG. 3D illustrates a photograph of assembling a bottom portion of a compression set-up of the DMA 850 instrument of FIGS. 3A-3B;

FIG. 3E illustrates a photograph of aligning a top clamp to the bottom clamp for the DMA instrument of FIGS. 3A-3B;

FIG. 3F illustrates a second screen shot of the TRIOS software package for the DMA 850 instrument of FIGS. 3A-3B and 3D-3E, showing a Clamp Calibration tab;

FIG. 3G-1 illustrates a screen shot of the DMA 850 instrument, showing a float icon;

FIG. 3G-2 illustrates a third screen shot of the TRIOS software package for the DMA 850 instrument of FIGS. 3A-3B and 3D-3E, showing sample dimensions;

FIG. 3G-3 illustrates a fourth screen shot of the TRIOS software package for the DMA 850 instrument of FIGS. 3A-3B and 3D-3E, showing a DMA Control tab;

FIG. 3H illustrates a graph of Strain ε (%) vs. Stress σε (kPa) of an advanced manufactured interpenetrating polymer network (AM-IPN) for Polyethylene Oxide (PEO) 4000000 in synthetic AA42, AL35 (0.5% PEO), AL36 (0.1% PEO), AA42, AL43 (0.5% PEO), AL44 (0.1% PEO), AL45 (0.05% PEO) and AI28 Bioinks and Lung G1, showing a comparison of PEO concentrations in the Bioinks;

FIG. 3I illustrates a graph of Strain ε (%) vs. Stress σε (kPa) of an advanced manufactured interpenetrating polymer network (AM-IPN) for Polyethylene Oxide (PEO) 4000000 in synthetic AL35, AL36 and AA42 Bioinks, showing a comparison of PEO concentrations in the Bioinks;

FIG. 3J illustrates a graph of Strain ε (%) vs. Stress σε (kPa) of an advanced manufactured interpenetrating polymer network (AM-IPN) for Polyethylene Oxide (PEO) in synthetic AL42, AL45, AI28 Bioinks and Lung G1, showing a comparison of PEO concentrations in the Bioinks;

FIG. 3K illustrates a graph of Strain ε (%) vs. Stress σε (kPa) of an advanced manufactured interpenetrating polymer network (AM-IPN) for Polyethylene Oxide (PEO) 4000000 in synthetic AL45 (3% Triacrylate, 0.05% PEO), AL48 (4% Triacrylate, 0.05% PEO), AL49 (3% Triacrylate, 0.01% PEO) Bioinks and Lung G1, showing a comparison of PEO concentrations in the Bioinks;

FIG. 4A illustrates a photograph of an advanced manufactured interpenetrating polymer network (AM-IPN) for Polyethylene Oxide (PEO) in synthetic AL35 Bioink;

FIG. 4B a photograph of an advanced manufactured interpenetrating polymer network (AM-IPN) for Polyethylene Oxide (PEO) in synthetic AL36 Bioink;

FIG. 4C a photograph of an advanced manufactured interpenetrating polymer network (AM-IPN) for Polyethylene Oxide (PEO) in synthetic AL37 Bioink;

FIG. 5 illustrates a graph of Strain ε (%) vs. Stress σε (kPa) of an advanced manufactured interpenetrating polymer network (AM-IPN) for Polyethylene Oxide (PEO) in synthetic AL45 (energy of 210 mJ), AL49 (energy of 90 mJ) and AI28 (Parent) (energy of 48 mJ) Bioinks;

FIG. 6A illustrates a photograph of an advanced manufactured interpenetrating polymer network (AM-IPN) for Polyethylene Oxide (PEO) in synthetic AL45 Bioink (3% Triacrylate, 0.05% PEO), showing a 100.100 dense ring having an average vasculature thickness of 95.4±8.7 μm;

FIG. 6B illustrates a photograph of an advanced manufactured interpenetrating polymer network (AM-IPN) for Polyethylene Oxide (PEO) in synthetic AL45 Bioink, showing a 50.80 dense ring having an average vasculature thickness of 48.8±10.1 μm;

FIG. 7A illustrates a photograph of an advanced manufactured interpenetrating polymer network (AM-IPN) in an advanced manufactured hydrogel material of synthetic AL49 Bioink (3% Triacrylate, 0.1% PEO), showing the hydrogel material being placed in a bioreactor to prepare for perfusion and ventilation;

FIG. 7B illustrates a photograph of the AM-IPN in the advanced manufactured hydrogel material of FIG. 7A, showing the hydrogel material after being perfused and during ventilation;

FIG. 8A illustrates a photograph of a biocompatibility test (glass control) of an advanced manufactured interpenetrating polymer network (AM-IPN) for Polyethylene Oxide (PEO) in synthetic AL36 Bioink;

FIG. 8B illustrates a photograph of a biocompatibility test (Day 1) of an advanced manufactured interpenetrating polymer network (AM-IPN) for Polyethylene Oxide (PEO) in synthetic AL36 Bioink, showing attachment of small airway epithelial cells (SAEC);

FIG. 8C illustrates a photograph of a biocompatibility test (Day 4) of an advanced manufactured interpenetrating polymer network (AM-IPN) for Polyethylene Oxide (PEO) in synthetic AL36 Bioink, showing decreased attachment of small airway epithelial cells (SAEC);

FIG. 9A illustrates a photograph of a suture test of an advanced manufactured interpenetrating polymer network (AM-IPN) for Polyethylene Oxide (PEO) in synthetic AL48 (4% Triacrylate, 0.05% PEO) Bioink, showing a high crosslinking density of the interpenetrating polymer network in the Bioink;

FIG. 9B illustrates a photograph of a suture test of an advanced manufactured interpenetrating polymer network (AM-IPN) for Polyethylene Oxide (PEO) in synthetic AL49 (3% Triacrylate, 0.1% PEO) Bioink, showing a moderate crosslinking density of the interpenetrating polymer network in the Bioink;

FIG. 10 illustrates a graph of Step Time ts (s) vs. Storage Modulus G′ (kPa) of an advanced manufactured interpenetrating polymer network (AM-IPN) for Polyethylenimine (PEI) in synthetic AL60, AL62, AL364, AL65 and AL66 Bioinks;

FIG. 11 illustrates a graph of Strain ε (%) vs. Stress σε (kPa) of an advanced manufactured interpenetrating polymer network (AM-IPN) for Polyethylenimine (PEI) 750,000 in synthetic AL60 (power of 50 mW/cm², time of 1900 ms), AL62 (power of 50 mW/cm², time of 750 ms), AL62 (power of 150 mW/cm², time of 500 ms), AL62 (power of 200 mW/cm², time of 500 ms) Bioinks and Lung G1;

FIG. 12A illustrates a photograph of an advanced manufactured interpenetrating polymer network (AM-IPN) for Polyethylenimine (PEI) in synthetic AL60 Bioink;

FIG. 12B a photograph of an advanced manufactured interpenetrating polymer network (AM-IPN) for Polyethylenimine (PEI) in synthetic AL61 Bioink;

FIG. 12C a photograph of an advanced manufactured interpenetrating polymer network (AM-IPN) for Polyethylenimine (PEI) in synthetic AL62 (power of 50 mW/cm²) Bioink, showing a low crosslinking density of the interpenetrating polymer network of PEI in the Bioink;

FIG. 12D a photograph of an advanced manufactured interpenetrating polymer network (AM-IPN) for Polyethylenimine (PEI) in synthetic AL62 (150 mW/cm²) Bioink, showing a low crosslinking density of the interpenetrating polymer network of PEI in the Bioink;

FIG. 12E a photograph of an advanced manufactured interpenetrating polymer network (AM-IPN) for Polyethylenimine (PEI) in synthetic AL62 (200 mW/cm²) Bioink, showing a low crosslinking density of the interpenetrating polymer network of PEI in the Bioink;

FIG. 13A illustrates a photograph of a suture test of an advanced manufactured interpenetrating polymer network (AM-IPN) for Polyethylenimine (PEI) in synthetic AL62 (power of 50 mW/cm², time of 750 ms) Bioink, showing a low crosslinking density of the interpenetrating polymer network of PEI in the Bioink;

FIG. 13B illustrates a photograph of a suture test of an advanced manufactured interpenetrating polymer network (AM-IPN) for Polyethylenimine (PEI) in synthetic AL62 (power of 100 mW/cm², time of 500 ms) Bioink, showing a low crosslinking density of the interpenetrating polymer network of PEI in the Bioink;

FIG. 13C illustrates a photograph of a suture test of an advanced manufactured interpenetrating polymer network (AM-IPN) for Polyethylenimine (PEI) in synthetic AL62 (power of 150 mW/cm², time of 450 ms) Bioink, showing moderate crosslinking density of the interpenetrating polymer network of PEI in the Bioink;

FIG. 13D illustrates a photograph of a suture test of an advanced manufactured interpenetrating polymer network (AM-IPN) for Polyethylenimine (PEI) in synthetic AL62 (power of 200 mW/cm², time of 400 ms) Bioink, showing moderate crosslinking density of the interpenetrating polymer network of PEI in the Bioink;

FIG. 14 illustrates a graph of Step Time ts (s) vs. Storage Modulus G′ (kPa) of an advanced manufactured interpenetrating polymer network (AM-IPN) for Polyvinylpyrrolidone (PVP) in synthetic AL68, AL69, AL70, AL72, AL73, AL74 and AL 75 Bioinks;

FIG. 15 illustrates a graph of Strain ε (%) vs. Stress σε (kPa) of an advanced manufactured interpenetrating polymer network (AM-IPN) for Polyvinylpyrrolidone (PVP) in synthetic AL68 (3% Triacrylate, 0.5% PVP), AL70 (3% Triacrylate, 0.05% PVP), AL72 (2% Triacrylate, 1% PVP), AL73 (2% Triacrylate, 0.5% PVP), AL74 (2% Triacrylate, 0.1% PVP) Bioinks and Lung G1;

FIG. 16A illustrates a photograph of an advanced manufactured interpenetrating polymer network (AM-IPN) for Polyvinylpyrrolidone (PVP) in synthetic AL68 Bioink;

FIG. 16B a photograph of an advanced manufactured interpenetrating polymer network (AM-IPN) for Polyvinylpyrrolidone (PVP) in synthetic AL70 Bioink;

FIG. 16C a photograph of an advanced manufactured interpenetrating polymer network (AM-IPN) for Polyvinylpyrrolidone (PVP) in synthetic AL72 Bioink;

FIG. 16D a photograph of an advanced manufactured interpenetrating polymer network (AM-IPN) for Polyvinylpyrrolidone (PVP) in synthetic AL73 Bioink;

FIG. 16E a photograph of an advanced manufactured interpenetrating polymer network (AM-IPN) for Polyvinylpyrrolidone (PVP) in synthetic AL74 Bioink;

FIG. 17A illustrates a photograph of a suture test of an advanced manufactured interpenetrating polymer network (AM-IPN) for Polyvinylpyrrolidone (PVP) in synthetic AL68 Bioink;

FIG. 17B illustrates a photograph of a suture test of an advanced manufactured interpenetrating polymer network (AM-IPN) for Polyvinylpyrrolidone (PVP) in synthetic AL70 Bioink;

FIG. 17C illustrates a photograph of a suture test of an advanced manufactured interpenetrating polymer network (AM-IPN) for Polyvinylpyrrolidone (PVP) in synthetic AL73 Bioink;

FIG. 18 illustrates a graph of Step Time ts (s) vs. Storage Modulus G′ (kPa) of an advanced manufactured interpenetrating polymer network (AM-IPN) for Polyvinyl Alcohol (PVA) in synthetic AL76 and AL 77 Bioinks;

FIG. 19 illustrates a graph of Strain ε (%) vs. Stress σε (kPa) of an advanced manufactured interpenetrating polymer network (AM-IPN) for Polyvinyl Alcohol (PVA) in synthetic AL76, AL77 Bioinks and Lung G1;

FIG. 20A illustrates a photograph of an advanced manufactured interpenetrating polymer network (AM-IPN) for Polyvinyl Alcohol (PVA) in synthetic AL76 (power of 50 mW/cm², time of 1000 ms) Bioink, showing a low crosslinking density of the interpenetrating polymer network of PVA in the Bioink;

FIG. 20B illustrates a photograph of an advanced manufactured interpenetrating polymer network (AM-IPN) for Polyvinyl Alcohol (PVA) in synthetic AL76 (power of 100 mW/cm², time of 700 ms) Bioink, showing a low to moderate crosslinking density of the interpenetrating polymer network of PVA in the Bioink;

FIG. 20C illustrates a photograph of an advanced manufactured interpenetrating polymer network (AM-IPN) for Polyvinyl Alcohol (PVA) in synthetic AL77 (power of 100 mW/cm², time of 700 ms) Bioink, showing a moderate crosslinking density of the interpenetrating polymer network of PVA in the Bioink;

FIG. 21A illustrates a photograph of an advanced manufactured interpenetrating polymer network (AM-IPN) for Polyvinyl Alcohol (PVA) in synthetic AL76 (power of 50 mW/cm², time of 1000 ms) Bioink, showing a low crosslinking density of the interpenetrating polymer network of PVA in the Bioink;

FIG. 21B illustrates a photograph of an advanced manufactured interpenetrating polymer network (AM-IPN) for Polyvinyl Alcohol (PVA) in synthetic AL76 (power of 50 mW/cm², time of 2500 ms) Bioink, showing a low to moderate crosslinking density of the interpenetrating polymer network of PVA in the Bioink;

FIG. 21C illustrates a photograph of an advanced manufactured interpenetrating polymer network (AM-IPN) for Polyvinyl Alcohol (PVA) in synthetic AL76 (power of 100 mW/cm², time of 700 ms) Bioink, showing a moderate crosslinking density of the interpenetrating polymer network of PVA in the Bioink;

FIG. 22A illustrates a photograph of a suture test of an advanced manufactured interpenetrating polymer network (AM-IPN) for Polyvinyl Alcohol (PVA) in synthetic AL76 (power of 100 mW/cm², time of 700 ms) Bioink, showing a moderate crosslinking density of the interpenetrating network of PVA in the Bioink;

FIG. 22B illustrates a photograph of a suture test of an advanced manufactured interpenetrating polymer network (AM-IPN) for Polyvinyl Alcohol (PVA) in synthetic AL77 (power of 100 mW/cm², time of 700 ms) Bioink, showing a moderate crosslinking density of the interpenetrating polymer network of PVA in the Bioink;

FIG. 23A illustrates a chart of Stress (kPa) for Polyvinylpyrrolidone (PVP) in synthetic AW55, AW99 and AI28 Bioinks;

FIG. 23B illustrates a chart of Strain (%) for Polyvinylpyrrolidone (PVP) in synthetic AW55, AW99 and AI28 Bioinks;

FIG. 23C illustrates a chart of Young's Modulus (kPa) for Polyvinylpyrrolidone (PVP) in synthetic AW55, AW99 and AI28 Bioinks; and

FIG. 24 illustrates a photograph of a hydrogel (dogbone) material.

### DETAILED DESCRIPTION

The following detailed description of various embodiments of the present invention references the accompanying drawings, which illustrate specific embodiments in which the invention can be practiced. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains. Therefore, the scope of the present invention is defined only by the appended claims, along with the full scope of equivalents to which such claims are entitled.

### Advanced Manufactured Interpenetrating Polymer Network (AM-IPN)

An interpenetrating polymer network is a primary network bonded to a secondary network via non-covalent bonds.

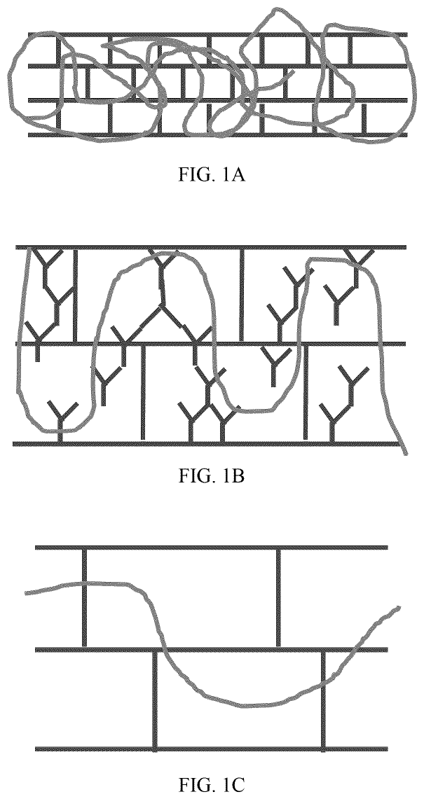

FIG. 1A illustrates an interpenetrating polymer network in a synthetic AA42 Bioink with 8% PEGDA 3400, showing a high crosslinking density of the interpenetrating polymer network in the Bioink.

FIG. 1B illustrates an interpenetrating polymer network in a synthetic AI28 Bioink with 5% PEGDA 6000 and 3% Triactetate, showing a moderate crosslinking density of the interpenetrating polymer network in the Bioink.

FIG. 1C illustrates an interpenetrating polymer network in a synthetic 602 Bioink with 5% PEGDA 6000, showing a low crosslinking density of the interpenetrating polymer network in the Bioink.

Any suitable synthetic bioink may be used. For example, suitable synthetic bioinks, include, but are not limited to, AA42, AI15, AI28, and AJ55 Bioinks, as described herein.

Exemplary Bioinks

AA42 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 3400 | 7.6-8.4 |
| LAP | 0.95-1.05 |
| UV386A | 0.114-0.126 |
| Water | 76.84-84.92 |
| Total | 100 |

AI15 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| HPA | 130.4 | HPA | 9.5-10.5 |
| | | 97699847GO | |
| | | 10Nov20 | |
| PEGDA | 6052 | PEGDA 6000 - | 4.75-5.25 |
| 6000 | | BB0308-78 | |
| TMPTA | 956 | SR9035 - | 0.95-1.05 |
| 912 | | LDJ1138 | |
| NAP | 311.26 | NAP -- | 1.43-1.58 |
| | | 0010769 | |
| UV386A | 478.03 | UV386A - | 0.152-0.168 |
| | | 0917-20A- | |
| | | 386A | |
| Water | | | 28.5-31.5 |
| Total | | | 100 |

AI28 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 2.85-3.15 |
| NAP | 1.52-1.68 |
| UV386A | 0.152-0.168 |
| Water | 76.23-84.25 |
| Total | 100 |

AJ55 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 1.9-2.1 |
| NAP | 0.95-1.05 |
| UV386A | 0.171-0.189 |
| Water | 76.78-84.86 |
| Total | 100 |

Any suitable polymer may be incorporated into a synthetic bioink. For example, suitable polymers include, but are not limited to, polyethylene oxide (PEG), Polyethylenimine (PEI), Polyvinylpyrrolidone (PVP), and Polyvinyl Alcohol (PVA).

Exemplary Polymers

| | Polyethylene | | | Polyvinyl |
|---|---|---|---|---|
| | Oxide | Polyethylenimine | Polyvinylpyrrolidone | Alcohol |
| | (PEO) | (PEI) | (PVP) | (PVA) |
| Molecular | 4000000, | 750000 | 1300000 | 93500 |
| Weight | 1000000, | 25000 | 1000000 | |
| (MW) | 100000 | | | |
| Solubility | 0.5% stock solution | 50% stock solution | 2% stock solution | 2% in stock solution |
| | Heat at 100° C., | No heat, mix at | No heat, mix at | Heat at 200° C., |
| | mix at 600 RPM | 600 RPM | 200 RPM | mix at 600 RPM |
| Compatibility | | Not compatible with | | |
| | | PEGDA 700 and | | |
| | | Triacetate | | |

Exemplary Bioink Ink Formulations and Print Settings for FS20 DLP 3D System Printer

Polyethylene Oxide (PEO)

In these examples, a Polyethylene Oxide (PEG) polymer was incorporated into AA42 Bioink (Stock), as follows.

Polyethylene Oxide 4000000 (0.5% PEG Stock Solution)

Polyethylene Oxide 4000000 polymer was added to water, heated at 100° C., and mixed at 600 RPM before incorporating the bioink.

Polyethylene Oxide 1000000 (0.5% PEO Stock Solution)

Polyethylene Oxide 1000000 polymer was added to water, heated at 100° C., and mixed at 600 RPM before incorporating into bioink.

AA42 Bioink (Stock)

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 3400 | 7.6-8.4 |
| LAP | 0.95-1.05 |
| UV386A | 0.114-0.126 |
| Water | 17.04-18.84 |
| Total | 35.21-38.91 |

In the AA42 Bioink (Stock) solution, the water concentration was reduced slightly (as compared to the AA42 Bioink) so that Polyethylene Oxide polymer stock solutions could be added to prepare the AL35, AL36, AL37, AL38 and AL39 Bioinks.

AL35 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEO | 4000000 | PEO 4000000 - | 0.48-0.53 |
| 4000000 | | MKCL3736 | |
| Water | | | 59.32-65.56 |
| AA42 | | | 35.21-38.91 |
| (Stock) | | | |
| Total | | | 100 |

AL36 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEO | 4000000 | PEO 4000000 - | 0.095-0.105 |
| 4000000 | | MKCL3736 | |
| Water | | | 59.70-65.98 |
| AA42 | | | 35.21-38.91 |
| (Stock) | | | |
| Total | | | 100 |

AL37 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEO | 4000000 | PEO 4000000 - | 0.048-0.053 |
| 4000000 | | MKCL3736 | |
| Water | | | 59.75-66.03 |
| AA42 | | | 35.21-38.91 |
| (Stock) | | | |
| Total | | | 100 |

AL38 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEO | 1000000 | PEO 1000000 - | 0.095-0.105 |
| 1000000 | | MKCM8338 | |
| Water | | | 59.75-66.03 |
| AA42 | | | 35.21-38.91 |
| (Stock) | | | |
| Total | | | 100 |

AL39 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEO | 1000000 | PEO 4000000 - | 0.048-0.053 |
| 1000000 | | MKCM8338 | |
| Water | | | 59.75-66.03 |
| AA42 | | | 35.21-38.91 |
| (Stock) | | | |
| Total | | | 100 |

AL42 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | | 4.75-5.25 |
| TAC | | 2.85-3.15 |
| NAP | | 1.52-1.68 |
| UV386A | | 0.152-0.168 |
| Water | | 75.75-83.73 |
| PEI 25000 | 25000 | 0.475-0.525 |
| Total | | 100 |

AL43 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | | 4.75-5.25 |
| TAC | | 0.95-1.05 |
| NAP | | 1.52-1.68 |
| UV386A | | 0.152-0.168 |
| Water | | 75.75-83.73 |
| PEO 40000000 | 4000000 | 0.048-0.053 |
| Total | | 100 |

AL44 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | | 4.75-5.25 |
| TAC | | 1.9-2.1 |
| NAP | | 1.52-1.68 |
| UV386A | | 0.152-0.168 |
| Water | | 75.75-83.73 |
| PEO 40000000 | 4000000 | 0.048-0.053 |
| Total | | 100 |

AL45 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | | 4.75-5.25 |
| TAC | | 2.85-3.15 |
| NAP | | 1.52-1.68 |
| UV386A | | 0.152-0.168 |
| Water | | 75.75-83.73 |
| PEO 40000000 | 4000000 | 0.048-0.053 |
| Total | | 100 |

AL47 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 4.75-5.25 |
| PEGDA 700 | 2.85-3.15 |
| PEGDA 6000 | 4.75-5.25 |
| NAP | 1.43-1.58 |
| UV386A | 0.152-0.168 |
| Water | 81.07-89.61 |
| Total | 100 |

AL48 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | | 4.75-5.25 |
| TAC | | 3.8-4.2 |
| NAP | | 1.52-1.68 |
| UV386A | | 0.152-0.168 |
| Water | | 75.75-83.73 |
| PEO 40000000 | 4000000 | 0.048-0.053 |
| Total | | 100 |

Rheology Testing

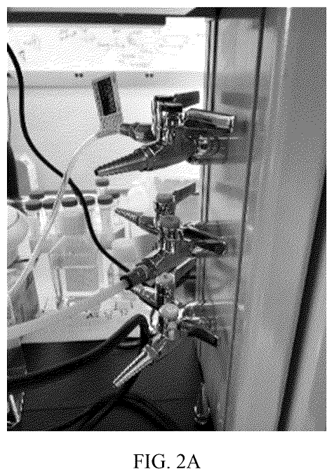

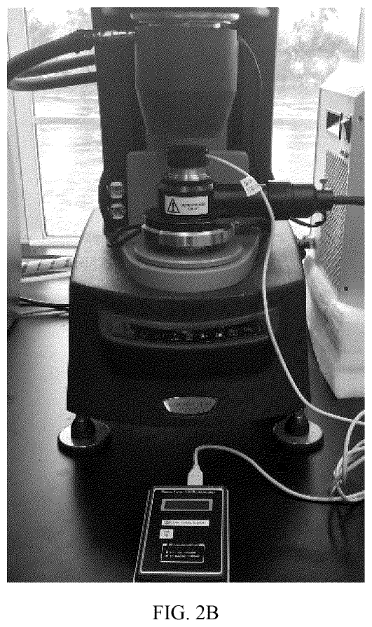

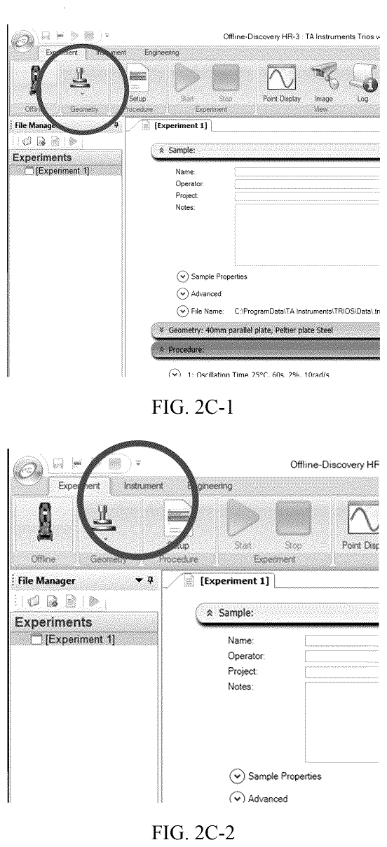

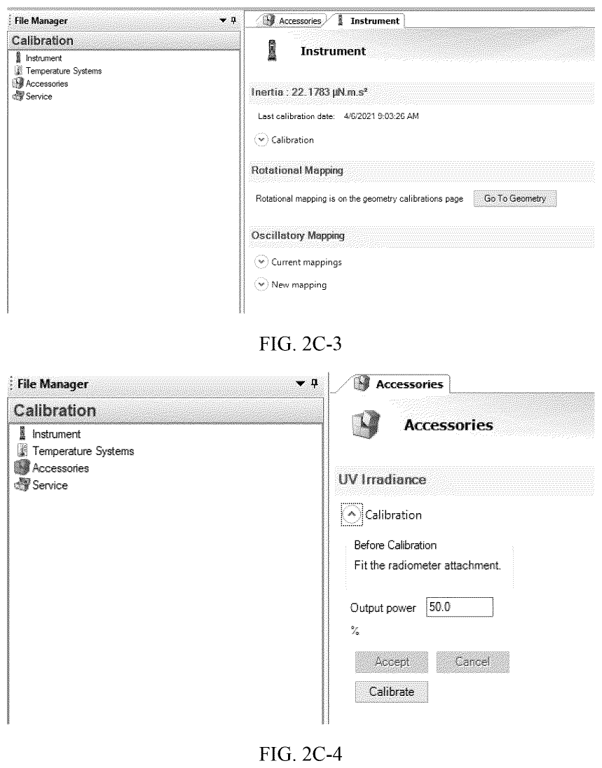

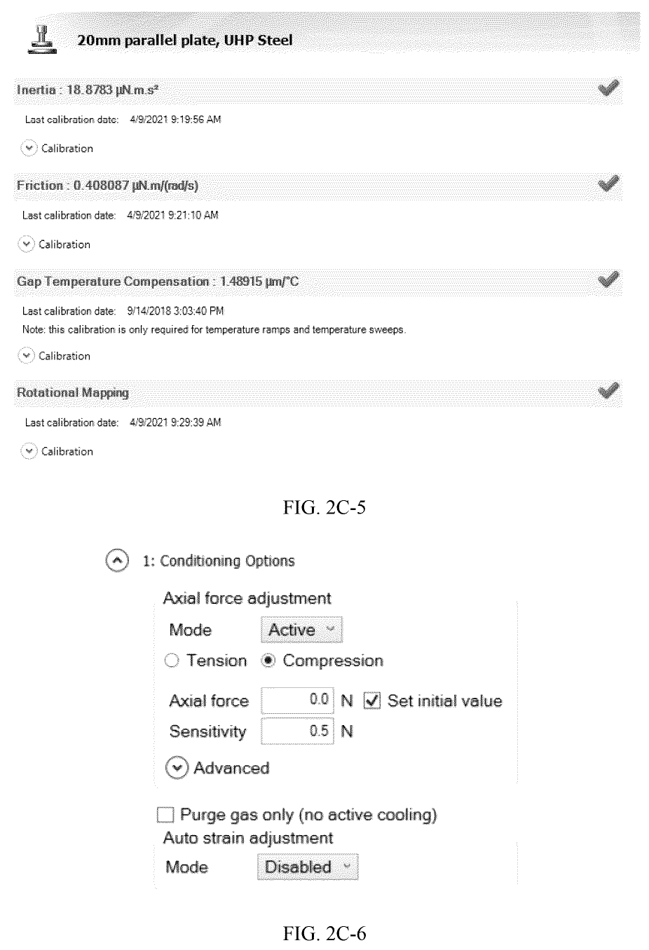

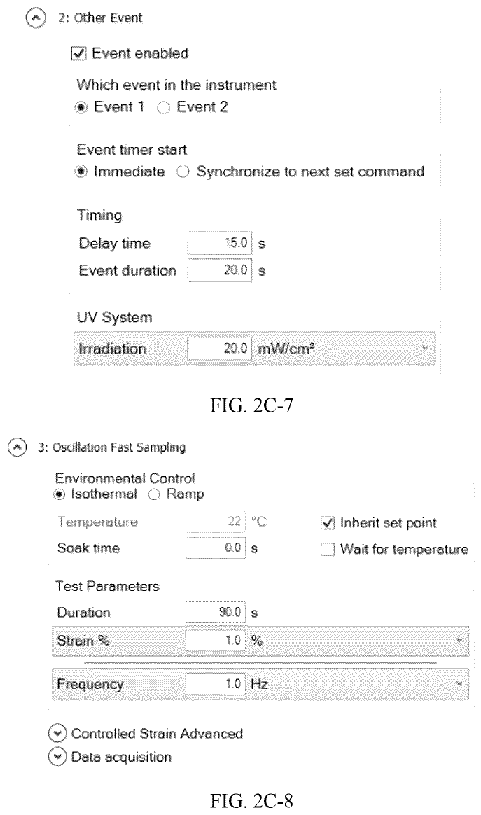

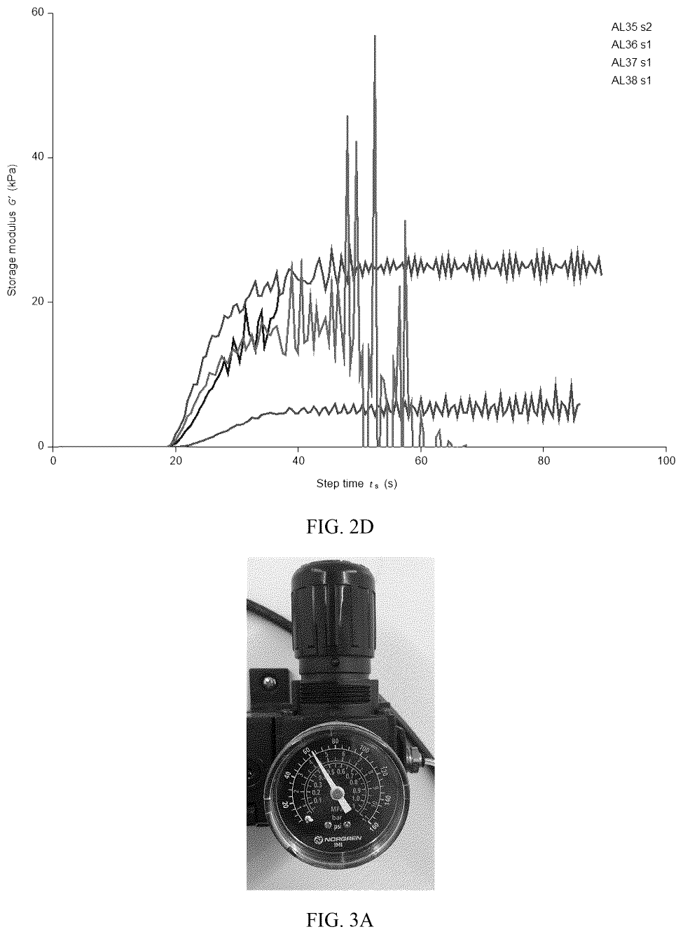

FIG. 2A illustrates a photograph of an air supply for a TA instrument with an OmniCure light source and a TRIOS software package. The air supply should be between about 20 psi and 40 psi air to the TA instrument.

FIG. 2B illustrates a photograph of the OmniCure light source for the TA instrument of FIG. 2A.

FIG. 2C-1 illustrates a first screen shot of the TRIOS software package for the TA instrument of FIGS. 2A-2B, showing a geometry icon under an experiment tab.

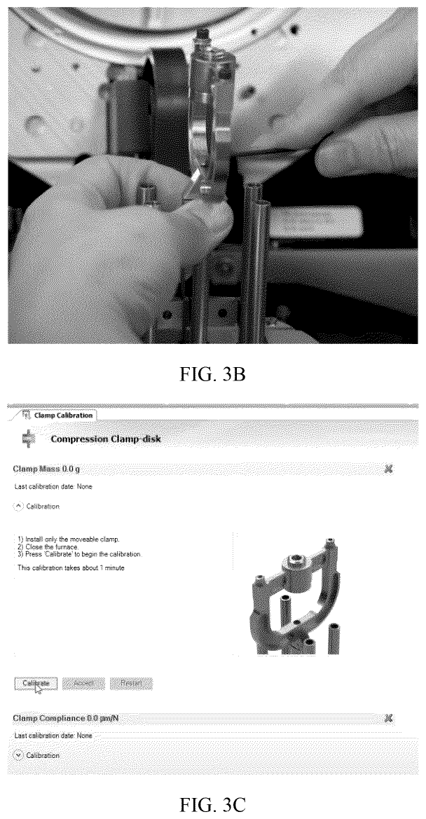

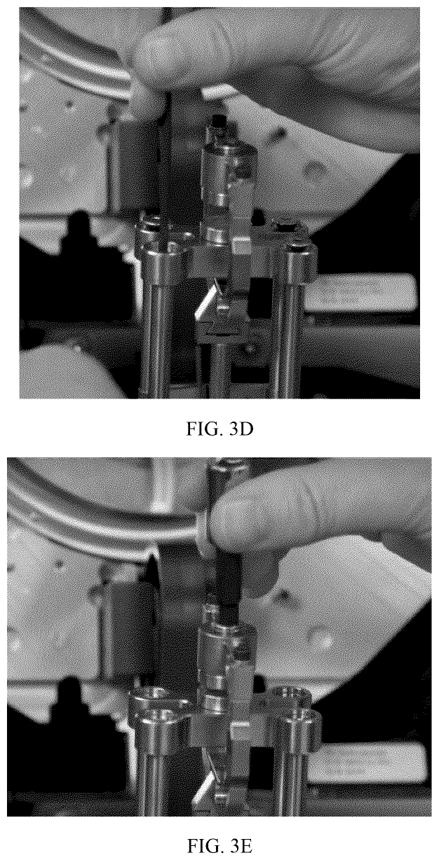

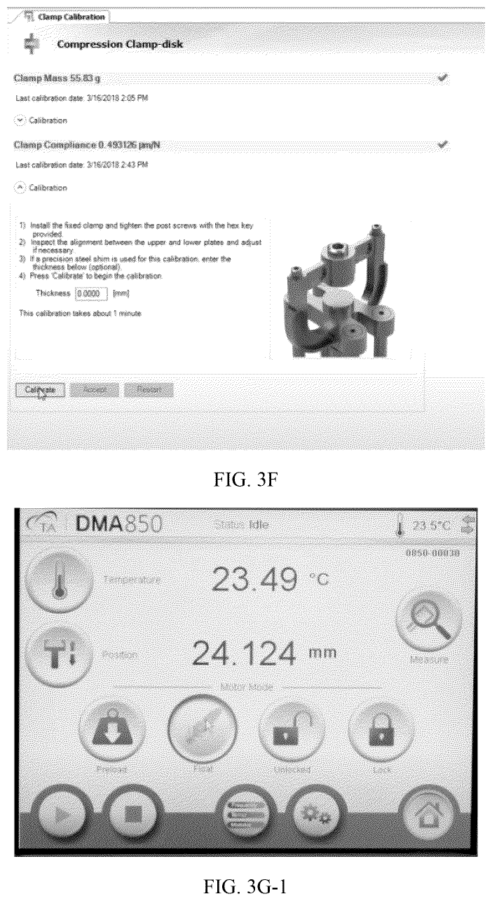

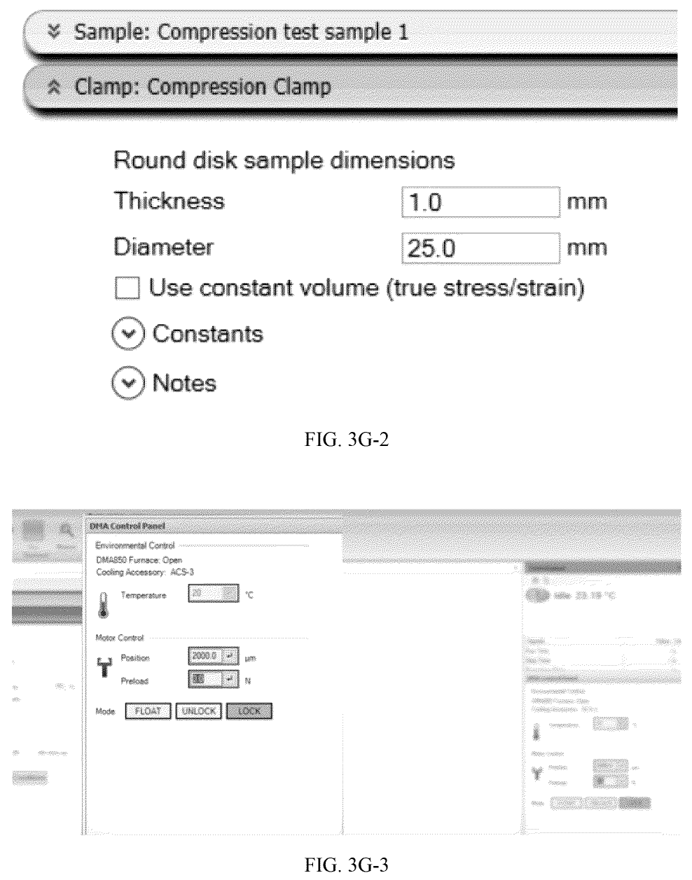

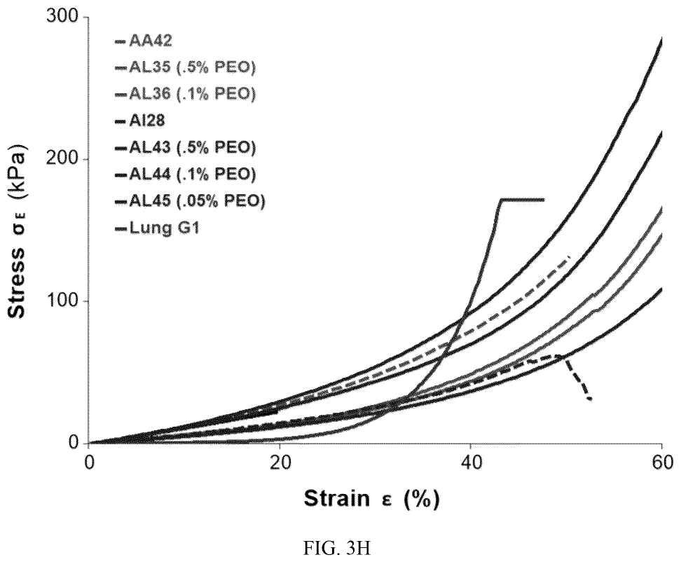

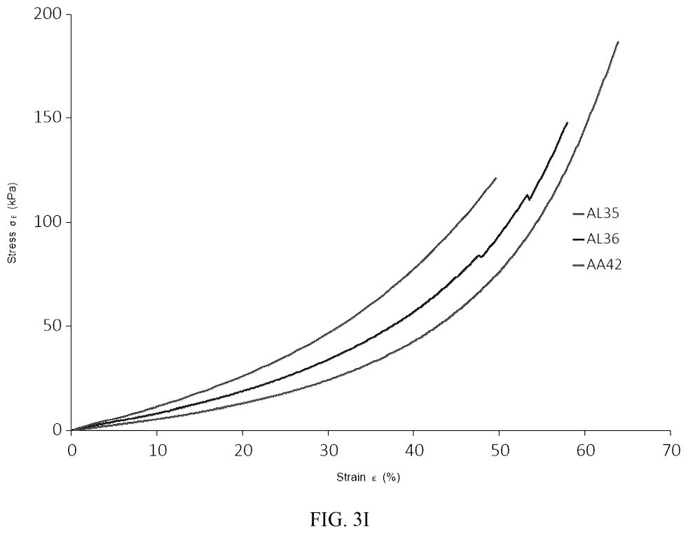

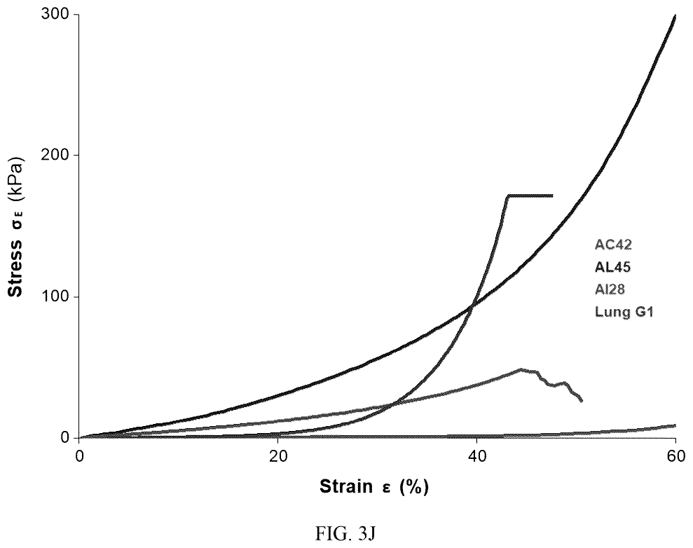

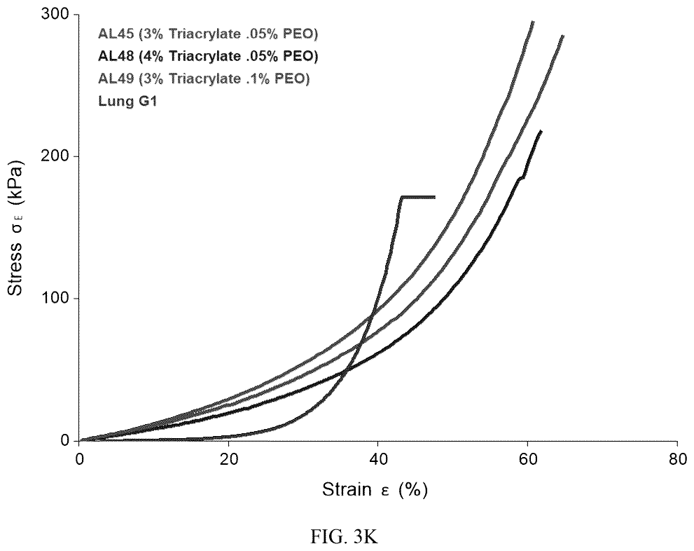

FIG. 2C-2 illustrates a second screen shot of the TRIOS software package for the TA instrument of FIGS. 2A-2B, showing an instrument tab; and FIG. 2C-3 illustrates a third screen shot of the TRIOS software package for the TA instrument of FIG. 2A, showing inertia (calibration) and oscillatory (new mapping) settings under the instrument tab.

Under the inertia settings, a new calibration reading was accepted because the reading was acceptable (e.g., close to previous calibration reading).

Under the oscillatory setting, a new mapping was calibrated.

The light source was calibrated with a black cover of a UV radiometer placed on the surface of the light source where a sample may be placed.

FIG. 2C-4 illustrates a fourth screen shot of the TRIOS software package for the TA instrument of FIGS. 2A-2B, showing accessories setting under a Calibration tab. The UV irradiation should be 50%. To calibrate the light source, the value measured by the UV radiometer was entered into the software (e.g., 56.2) and accepted.

FIG. 2C-5 illustrates a fifth screen shot of the TRIOS software package for the TA instrument of FIGS. 2A-2B, showing a Geometries tab. A geometry of 20 mm parallel plate was selected. A appropriate plate was placed onto the rheometer.

Under the inertia setting, a new calibration reading was accepted because the reading was acceptable (e.g., close to the previous reading)

Under the friction setting, a new calibration reading was accepted because the reading was acceptable (e.g., close to the previous reading).

The rheometer was lowered so that it was slightly above the sample placement and select a zero gap icon.

The rheometer was raised to a loading gap.

Under the Geometries tab, the gap was set to 200 μm and the minimum sample volume was set to 0.062 mL.

FIG. 2C-6 illustrates a sixth screen shot of the TRIOS software package of the TA instrument of FIGS. 2A-2B, showing condition options and settings.

FIG. 2C-7 illustrates a seventh screen shot of the TRIOS software package of the TA instrument of FIGS. 2A-2B, showing other event UV settings.

FIG. 2C-8 illustrates an eighth screen shot of the TRIOS software package of the TA instrument of FIGS. 2A-2B, showing oscillation fast sampling settings.

75 μL of sample was loaded onto the middle of the rheometry plate. Any bubble were removed by gently poking the bubbles with a needle. The gap was set to 200 μm. The black UV cover was lowered before each experiment to shield from UV light.

FIG. 2D illustrates a graph of Step Time ts (s) vs. Storage Modulus G′ (kPa) of an advanced manufactured interpenetrating polymer network (AM-IPN) for Polyethylene Oxide (PEO) in synthetic AL35, AL36, AL37 and AL38 Bioinks.

AL35, AL36, AL37 and AL38 Bioinks Rheology Data

| Bioink | Onset Time (s) | Storage Modulus at 50 s |
|---|---|---|
| AL35 | 20 | 4.42 |
| AL36 | 18.5 | 14.52 |
| AL37 | 17.5 | 16.73 |
| AL38 | 19 | 13.18 |

AL35, AL36, AL37 and AL38 Bioinks Print
Settings for FS20 DLP 3D System Printer

| | | | Region | | |
|---|---|---|---|---|---|
| Baselayer | B | Units | Layer | E | Units |
| Power | 50 | mW/cm² | Power | 50 | mW/cm² |
| Exposure | 1000 | ms | Exposure | 600 | ms |
| Time | | | Time | | |
| Pump | 300 | μm | Pump | 300 | μm |
| Distance | | | Distance | | |
| Uptime | 1000 | ms | Uptime | 1500 | ms |
| Uptime | 1000 | ms | Uptime | 1500 | ms |
| Pause | | | Pause | | |
| Downtime | 1000 | ms | Downtime | 1500 | ms |
| Downtime | 1000 | ms | Down time | 2000 | ms |
| Pause | | | Pause | | |

Compression Testing

Dynamic Mechanical Analysis (DMA) is a technique that is frequently used to characterize a material's properties as a function of atmosphere, frequency, stress, temperature, time, and combinations thereof.

The compression testing is performed using a DMA 850 instrument designed to measure viscoelastic properties (e.g., modulus, damping) of rigid and soft solid materials. The compression testing may be used to measure the properties of low to medium modulus materials, including gels and weak elastomers. The sample must support a static (preload) force during compression testing. The sample should have as high of a thickness-to-diameter ratio as possible, depending on the sample preparation and instrument limits.

Typical samples are printed discs (e.g., about 8 mm in diameter, about 3 mm thickness).

A printed disc is mounted in a clamp, one part of which is stationary and the other part is connected to a drive motor, and is movable. The motor directly applies a deformation to the printed disc.

The compression modulus is calculated from the slope of the stress (kPa) to the strain (%) in a linear region, which is typically from 0% to 10% of the initial strain. To ensure that the slope is calculated over the linear region, an R² value of the modulus should be between about 0.95 to about 0.99.

Compression failure strength and strain are the values of maximum stress (kPa) and maximum strain (%) at which the printed disc fails.

FIG. 3A illustrates an air supply for a DMA 850 instrument with a TRIOS software package. The air supply should be about 60 psi to the DMA instrument.

FIG. 3B illustrates a photograph of calibrating a bottom clamp for the DMA 850 instrument of FIG. 3A. The bottom clamp for the DMA instruments should be calibrated before the compression testing. The bottom clamp is mounted onto the DMA instrument and tightened with an Allen key until the bottom clamp is aligned and stable.

FIG. 3C illustrates a first screen shot of the TRIOS software package for the DMA 850 instrument of FIGS. 3A-3B, showing a Clamp Calibration tab. The furnace of the DMA instrument was closed. The mass of the bottom clamp was calibrated and accepted.

FIG. 3D illustrates a photograph of assembling a bottom portion of a compression set-up of the DMA 850 instrument of FIGS. 3A-3B. After the mass of the bottom clamp was calibrated, the bottom portion of the compression set-up was assembled and tightened in place with the Allen key.

FIG. 3E illustrates a photograph of aligning a top clamp to the bottom clamp for the DMA instrument of FIGS. 3A-3B. After a bottom plate was installed, the top clamp was aligned to the bottom clamp and tightened in place using an Allen key.

FIG. 3F illustrates a second screen shot of the TRIOS software package for the DMA 850 instrument of FIGS. 3A-3B and 3D-3E, showing a Clamp Calibration tab. Once the plates are perfectly aligned, the clamp was calibrated for compliance and accepted. The compliance value should be less to 1 μN/m to be acceptable.

FIG. 3G-1 illustrates a screen shot of the DMA 850 instrument, showing a float icon. After setting up the clamps, the clamps should be calibrated. The float icon is pressed to float the top clamp. This makes sure that the top clamp is gentle on the printed disc.

The printed discs should be stored in 1×DPBS overnight before the compression testing. Immediately before placing the printed discs in the clamps excess 1×DPBS should be gently blotted away using a Kimwipe.

FIG. 3G-2 illustrates a third screen shot of the TRIOS software package for the DMA 850 instrument of FIGS. 3A-3B and 3D-3E, showing sample dimensions. The diameter and thickness of the printed disc is measured using a Vernier caliper. The measurements are entered into the TRIOS software package.

The printed disc is placed between the bottom and top clamps, making sure that the printed disc is placed in the center between the clamps. The movable top clamp is brought down to slightly touch the printed disc.

Under a Procedure tab, the desired compression parameters (e.g., final strain percentage, strain rate) should be entered. Typical compression tests are performed at about 20% strain/minute.

FIG. 3G-3 illustrates a fourth screen shot of the TRIOS software package for the DMA 850 instrument of FIGS. 3A-3B and 3D-3E, showing a DMA Control tab. A preload force of 0.05 N for the printed disc (e.g., hydrogel) was entered. The compression testing may be run with an open furnace. Once the printed disc begins to fail (e.g., break), the compression testing may be stopped.

After the compression testing is stopped, Young's modulus may be calculated from the stress-strain curve slop in the linear region (e.g., about 0% to about 10% strain). The ultimate compressive strain (%) and strength (kPa) may be calculated at the failure point. An average of about six printed discs should be calculated for Young's modulus and failure strain.

FIG. 3H illustrates a graph of Strain ε (%) vs. Stress σε (kPa) of an advanced manufactured interpenetrating polymer network (AM-IPN) for Polyethylene Oxide (PEO) 4000000 in synthetic AA42, AL35 (0.5% PEO), AL36 (0.1% PEO), AA42, AL43 (0.5% PEO), AL44 (0.1% PEO), AL45 (0.05% PEO) and AI28 Bioinks and Lung G1, showing a comparison of PEO concentrations in the Bioinks.

FIG. 3I illustrates a graph of Strain ε (%) vs. Stress σε (kPa) of an advanced manufactured interpenetrating polymer network (AM-IPN) for Polyethylene Oxide (PEO) 4000000 in synthetic AL35, AL36 and AA42 Bioinks, showing a comparison of PEO concentrations in the Bioinks.

FIG. 3J illustrates a graph of Strain ε (%) vs. Stress σε (kPa) of an advanced manufactured interpenetrating polymer network (AM-IPN) for Polyethylene Oxide (PEO) in synthetic AL42, AL45, AI28 Bioinks and Lung G1, showing a comparison of PEO concentrations in the Bioinks.

FIG. 3K illustrates a graph of Strain ε (%) vs. Stress σε (kPa) of an advanced manufactured interpenetrating polymer network (AM-IPN) for Polyethylene Oxide (PEO) 4000000 in synthetic AL45 (3% Triacrylate, 0.05% PEO), AL48 (4% Triacrylate, 0.05% PEO), AL49 (3% Triacrylate, 0.01% PEO) Bioinks and Lung G1, showing a comparison of PEO concentrations in the Bioinks.

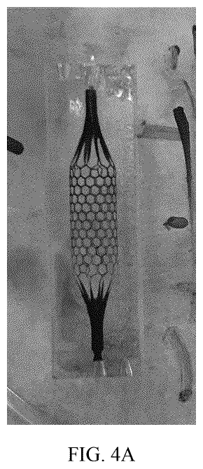

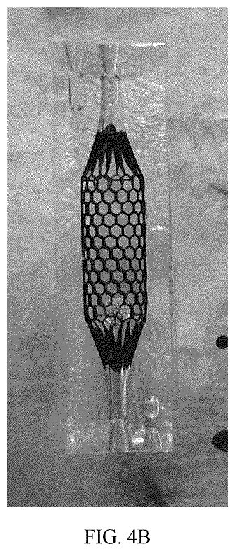

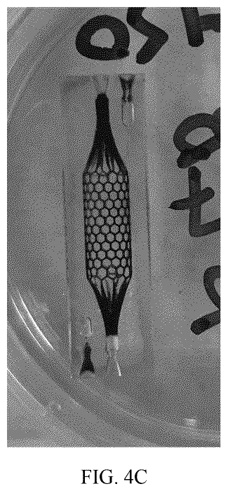

FIG. 4A illustrates a photograph of an advanced manufactured interpenetrating polymer network (AM-IPN) for Polyethylene Oxide (PEO) in synthetic AL35 Bioink.

FIG. 4B a photograph of an advanced manufactured interpenetrating polymer network (AM-IPN) for Polyethylene Oxide (PEO) in synthetic AL36 Bioink.

FIG. 4C a photograph of an advanced manufactured interpenetrating polymer network (AM-IPN) for Polyethylene Oxide (PEO) in synthetic AL37 Bioink.

AL35 Bioink Compression Data

| Area Under Curve | % Maximum Strain |
|---|---|
| 2945.1 | 188.91 |
| 2751.15 | 170.74 |
| 3228.81 | 199.01 |
| 2152.97 | 134.12 |
| 2769.71 | 173.20 |

AL36 Bioink Compression Data

| Area Under Curve | % Maximum Strain |
|---|---|
| 4910.25 | 308.44 |
| 2481.46 | 145.35 |
| 2655.55 | 150.95 |
| 3010.47 | 193.83 |
| 3078.87 | 181.54 |
| 2834.68 | 165.00 |
| 3161.88 | 190.85 |

FIG. 5 illustrates a graph of Strain ε (%) vs. Stress σε (kPa) of an advanced manufactured interpenetrating polymer network (AM-IPN) for Polyethylene Oxide (PEO) in synthetic AL45 (energy of 210 mJ), AL49 (energy of 90 mJ) and A128 (Parent) (energy of 48 mJ) Bioinks.

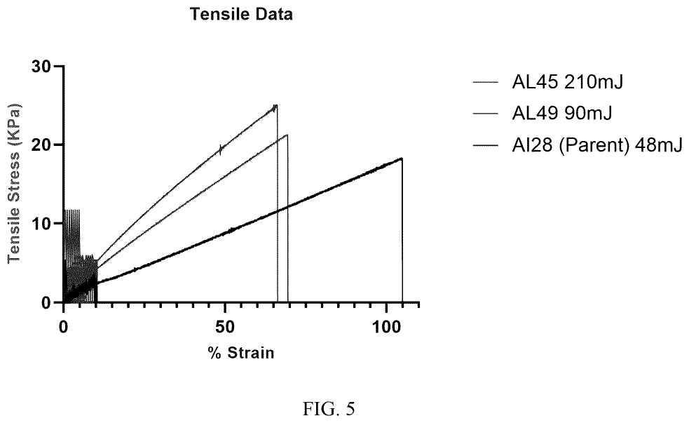

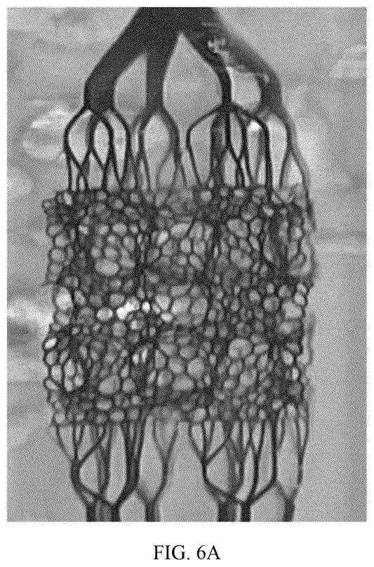

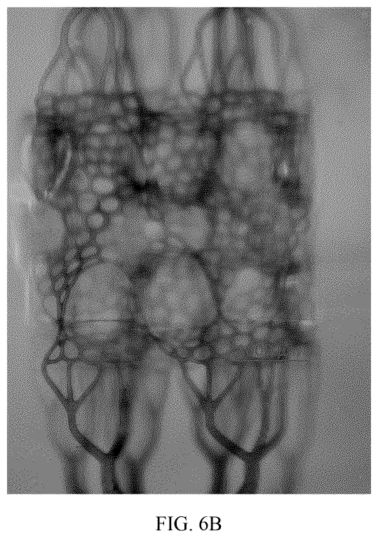

FIG. 6A illustrates a photograph of an advanced manufactured interpenetrating polymer network (AM-IPN) for a Polyethylene Oxide (PEO) in synthetic AL45 Bioink (3% Triacrylate, 0.05% PEO 4000000), showing a 100.100 dense ring having an average vasculature thickness of 95.4±8.7 μm.

FIG. 6B illustrates a photograph of an advanced manufactured interpenetrating polymer network (AM-IPN) for a Polyethylene Oxide (PEO) in synthetic AL45 Bioink, showing a 50.80 dense ring having an average vasculature thickness of 48.8±10.1 μm.

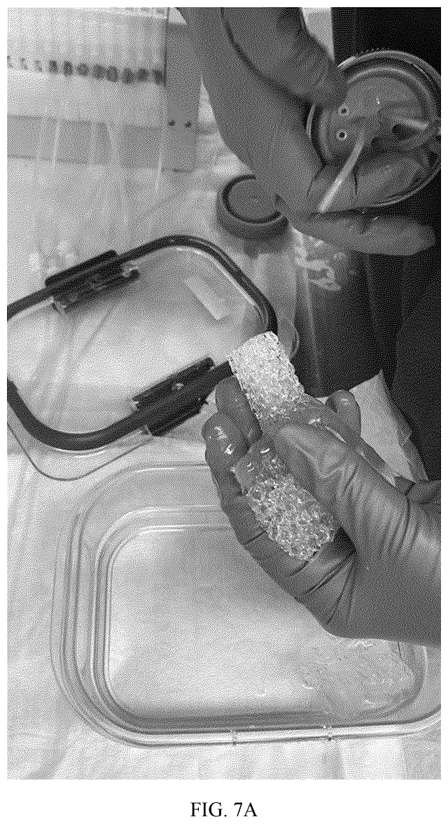

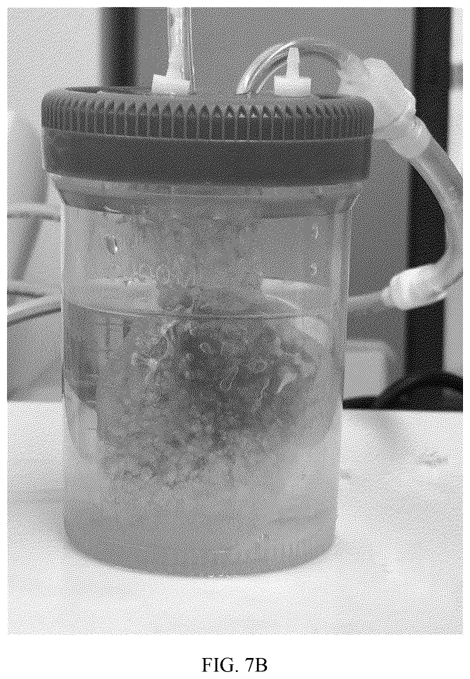

FIG. 7A illustrates a photograph of an advanced manufactured interpenetrating polymer network (AM-IPN) in an advanced manufactured hydrogel material of synthetic AL49 Bioink (3% Triacrylate, 0.1% PEO 4000000), showing the hydrogel material being placed in a bioreactor to prepare for perfusion and ventilation.

AL49 Bioink

| | | | | |
|---|---|---|---|---|
| | Molecular | Registry | Stock | |
| Reagent | Weight (MW) | No. | Solution (%) | Amount (%) |

| | | |
|---|---|---|
| HPA | | 9.5-10.5 |
| PEGDA 6000 | 6000 | 4.75-5.25 |
| TAC | | 2.85-3.15 |
| PEO 4000000 | 4000000 | 0.095-0.105 |
| NAP | | 1.52-1.68 |
| UV386A | | 0.152-0.168 |
| Water | | 76.09-84.09 |
| Total | | 100 |

The hydrogel material is placed in a bioreactor; and the tubes for the vasculature and airway are inserted from a blue cap of the bioreactor. This tests the ability of the hydrogel material to expand around the tube.

FIG. 7B illustrates a photograph of the AM-IPN in the advanced manufactured hydrogel material of FIG. 7A, showing the hydrogel material after being perfused and during ventilation.

Biocompatibility Testing

Printed discs (e.g., hydrogels) of AL36 Bioink were printed (e.g., about 8 cm diameter, about 3 mm thickness).

Small airway epithelial cells were seeded on the printed discs (e.g., hydrogels). This was done in clear bottom cell culture plates.

At each time point, the printed disks e.g., hydrogels) were placed under a microscope to determine whether small airway epithelial cell attachment was still occurring and whether the small airway epithelial cells remained unstained.

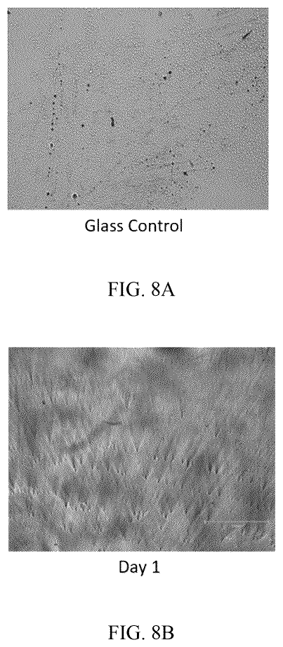

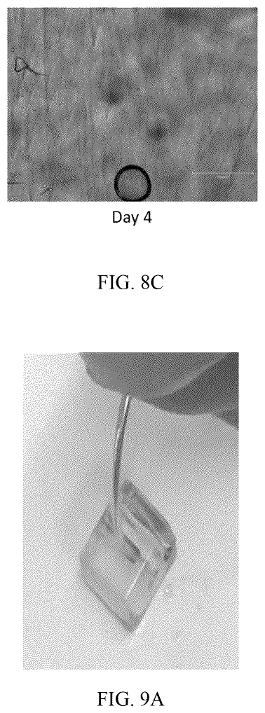

FIG. 8A illustrates a photograph of a biocompatibility test (glass control) for Polyethylene Oxide (PEO) in synthetic AL36 Bioink.

FIG. 8B illustrates a photograph of a biocompatibility test (Day 1) of an advanced manufactured interpenetrating polymer network (AM-IPN) for Polyethylene Oxide (PEO) in synthetic AL36 Bioink, showing attachment of small airway epithelial cells (SAEC).

FIG. 8C illustrates a photograph of a biocompatibility test (Day 4) of an advanced manufactured interpenetrating polymer network (AM-IPN) for Polyethylene Oxide (PEO) in synthetic AL36 Bioink, showing decreased attachment of small airway epithelial cells (SAEC).

Suture Testing

Using a 45 mm gauge needle, the needle was passed through a solid part of an advanced manufactured hydrogel material (capsulent). The hydrogel material (capsulent) was inspected for any cracking. The purpose of this test was to see if the hydrogel material could be sutured without cracking or falling apart.

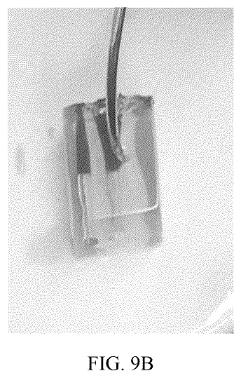

FIG. 9A illustrates a photograph of a suture test of an advanced manufactured interpenetrating polymer network (AM-IPN) for Polyethylene Oxide (PEO) in synthetic AL48 (4% Triacrylate, 0.05% PEO) Bioink, showing a high crosslinking density of the interpenetrating polymer network in the Bioink.

FIG. 9B illustrates a photograph of a suture test of an advanced manufactured interpenetrating polymer network (AM-IPN) for Polyethylene Oxide (PEO) in synthetic AL49 (3% Triacrylate, 0.1% PEO) Bioink, showing a moderate crosslinking density of the interpenetrating polymer network in the Bioink.

The AL35 Bioink swelled and disintegrated during surface modification. However, the AL36 Bioink remained intact during surface modification but showed decreased attachment of small airway epithelial cells (SAEC) by Day 4.

The AA42 Bioink forms a high crosslinking density of the interpenetrating polymer network in the Bioink. As such, the Polyethylene Oxide (PEO) polymer may be tightly bonded in the interpenetrating polymer network.

Polyethylenimine (PEI)

In these examples, a Polyethylenimine (PEI) polymer was incorporated into AI28 Bioink (Stock), as follows.

Polyethylenimine (PEI) 750000 (50% PEI Stock Solution)

Polyethylenimine (PEI) 750000 was dissolved in water, and mixed at 600 RPM before adding other components.

Polyethylenimine (PEI) 25000 (50% PEI Stock Solution)

Polyethylenimine (PEI) 25000 was dissolved in water, and mixed at 600 RPM before adding other components

AI28 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 2.85-3.15 |
| NAP | 1.52-1.68 |
| UV386A | 0.152-0.168 |
| Water | 76.23-84.25 |
| Total | 100 |

AL60 Bioink (Stock)

| | | | | |
|---|---|---|---|---|
| | Molecular | Registry | Stock | |
| Reagent | Weight (MW) | No. | Solution (%) | Amount (%) |

| | | | |
|---|---|---|---|
| HPA | 130.14 | HPA -- | 9.5-10.5 |
| | | 769984760 | |
| PEGDA | 6052 | PEGDA 6000 - | 4.75-5.25 |
| 6000 | | BB0308-178 | |
| | | PEGDA 6000 - | |
| | | BB0308-180 | |
| | | PEGDA 6000 - | |
| | | BB0308-181 | |
| PEGDA | 3452 | PEGDA 3400 - | 2.85-3.15 |
| 3400 | | BB0308-73 | |
| | | PEGDA 3400 - | |
| | | BB0308-73 | |
| | | PEGDA 3400 - | |
| | | BB0308-90 | |
| NAP | 311.26 | NAP -- | 1.43-1.58 |
| | | 0010769 | |
| UV386A | 478.03 | UV386A 903- | 0.114-0.126 |
| | | 20A-386A | |
| Water | | | 28.5-31.5 |
| Total | | | 50 |

AL60 Bioink

| | | | | |
|---|---|---|---|---|
| | Molecular | Registry | Stock | |
| Reagent | Weight (MW) | No. | Solution (%) | Amount (%) |

| | |
|---|---|
| Water | 47.86-52.90 |
| AL60 (Stock) | 47.14-52.10 |
| Total | 100 |

AL61 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 750000 | 750000 | PEI 750000 - | 0.048-0.053 |
| | | BCC0334 | |
| Water | | | 47.81-52.85 |
| AL60 | | | 47.14-52.10 |
| (Stock) | | | |
| Total | | | 100 |

AL62 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 750000 | 750000 | PEI 750000 - | 0.095-0.105 |
| | | BCC0334 | |
| Water | | | 47.77-52.79 |
| AL60 | | | 47.14-52.10 |
| (Stock) | | | |
| Total | | | 100 |

AL63 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 750000 | 750000 | PEI 750000 - | 0.475-0.525 |
| | | BCC0334 | |
| Water | | | 47.39-52.37 |
| AL60 | | | 47.14-52.10 |
| (Stock) | | | |
| Total | | | 100 |

AL64 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 25000 | 25000 | PEI 25000 - | 0.095-0.105 |
| | | 408727 | |
| Water | | | 47.77-52.79 |
| AL60 | | | 47.14-52.10 |
| (Stock) | | | |
| Total | | | 100 |

AL65 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 25000 | 25000 | PEI 25000 - | 0.048-0.053 |
| | | 408727 | |
| Water | | | 47.81-52.85 |
| AL60 | | | 47.14-52.10 |
| (Stock) | | | |
| Total | | | 100 |

AL66 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PEI 25000 | 25000 | PEI 25000 - | 0.475-0.525 |
| | | 408727 | |
| Water | | | 47.39-52.37 |
| AL60 | | | 47.17-52.10 |
| (Stock) | | | |
| Total | | | 100 |

Rheology Testing

The rheology testing discussed above and shown in FIGS. 2A-2C-8.

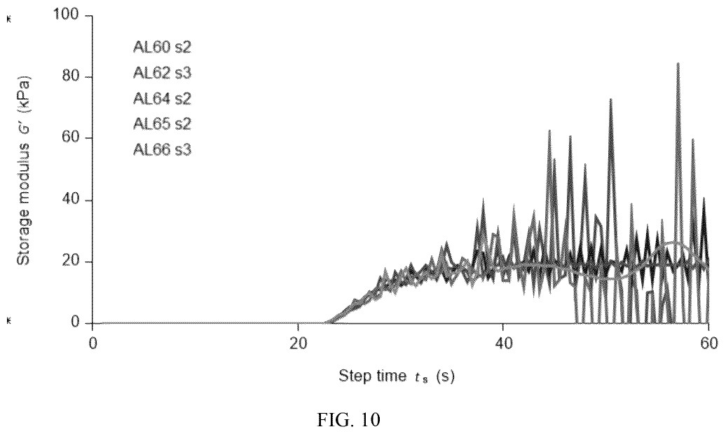

FIG. 10 illustrates a graph of Step Time ts (s) vs. Storage Modulus G′ (kPa) of an advanced manufactured interpenetrating polymer network (AM-IPN) for Polyethylenimine (PEI) in synthetic AL60, AL62, AL364, AL65 and AL66 Bioinks.

AL60, AL62, AL64, AL65 and AL66 Bio ink Rheology Data

| Bioink | Onset Time | Storage Modulus at 50 s |
|---|---|---|
| AL60 | 2.5 | 12.81 |
| AL62 | 2.5 | 12.39 |
| AL64 | 2.5 | 20.21 |
| AL65 | 2.5 | 16.79 |
| AL66 | 2.5 | 18.92 |

AL60 Bioink Print Settings for FS20 DLP 3D System Printer

| | | | Region | | |
|---|---|---|---|---|---|
| Baselayer | B | Units | Layer | E | Units |
| Power | 50 | mW/cm² | Power | 50 | mW/cm² |
| Exposure | 1500 | ms | Exposure | 1200 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

AL61 Bioink Print Settings for FS20 DLP 3D System Printer

| | | | Region | | |
|---|---|---|---|---|---|
| Baselayer | B | Units | Layer | E | Units |
| Power | 50 | mW/cm² | Power | 50 | mW/cm² |
| Exposure | 1000 | ms | Exposure | 800 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

AL62 (50 mW/cm²) Bioink Print Settings
for FS20 DLP 3D System Printer

| | | | Region | | |
|---|---|---|---|---|---|
| Baselayer | B | Units | Layer | E | Units |
| Power | 50 | mW/cm² | Power | 50 | mW/cm² |
| Exposure | 100 | ms | Exposure | 7500 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

AL62 (150 mW/cm²) Bioink Print Settings
for FS20 DLP 3D System Printer

| | | | Region | | |
|---|---|---|---|---|---|
| Baselayer | B | Units | Layer | E | Units |
| Power | 150 | mW/cm² | Power | 150 | mW/cm² |
| Exposure | 1000 | ms | Exposure | 500 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

AL62 (200 mW/cm²) Bioink Print Settings
for FS20 DLP 3D System Printer

| | | | Region | | |
|---|---|---|---|---|---|
| Baselayer | B | Units | Layer | E | Units |
| Power | 200 | mW/cm² | Power | 200 | mW/cm² |
| Exposure | 1000 | ms | Exposure | 500 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

Swelling Testing

Printed discs (e.g., hydrogels) of AL36 Bioink were printed (e.g., about 8 cm diameter, about 3 mm thickness).

The printed discs were weighed before soaking (i.e., initial weight). Immediately before weighing, any excess resin should be gently blotted from the printed disc using a Kimwipe.

The printed discs were placed in a 24-well plate. 1×DPBS++ was added to each well to about 5× the volume of the printed discs (i.e., a 8 mm×3 mm printed disc has a volume of about 150 μL, thus, about 1 mL of 1×DPBS++ should be added to each well).

The printed discs should be weighed after soaking in 1×DPBS++ for 24-hours at 37° C. (i.e., final weight after 24-hours). Immediately before weighing, any excess 1×DPBS++ should be gently blotted from the printed disc using a Kimwipe.

The % swelling may be calculated, as follows:

$$\% swelling = \frac{Final Weight ( mg ) - Initial Weight ( mg )}{Initial Weight ( mg )} * 100$$

Fresh 1×DPBS++ was added to each well. To continue the swelling testing, the printed discs should be weighed again after soaking in 1×DPBS++ for up to 4 or 7 days at 37° C. (i.e., final weight after 4 or 7 days).

AL62 Bioink Swelling Data at 37° C.

| Initial Weight | 24 Hours | Final Weight After 7 Days |
|---|---|---|
| (g) | (g) | (g) |
| 0.178 | 0.211 | 0.205 |
| 0.171 | 0.198 | 0.192 |
| 0.172 | 0.193 | 0.194 |
| 0.178 | 0.199 | 0.205 |
| 0.182 | 0.203 | 0.198 |
| 0.174 | 0.197 | 0.195 |
| 0.188 | 0.209 | 0.210 |
| 0.183 | 0.200 | 0.173 |

Compression Testing

The compression testing is discussed above and shown in FIGS. 3A-3G-3.

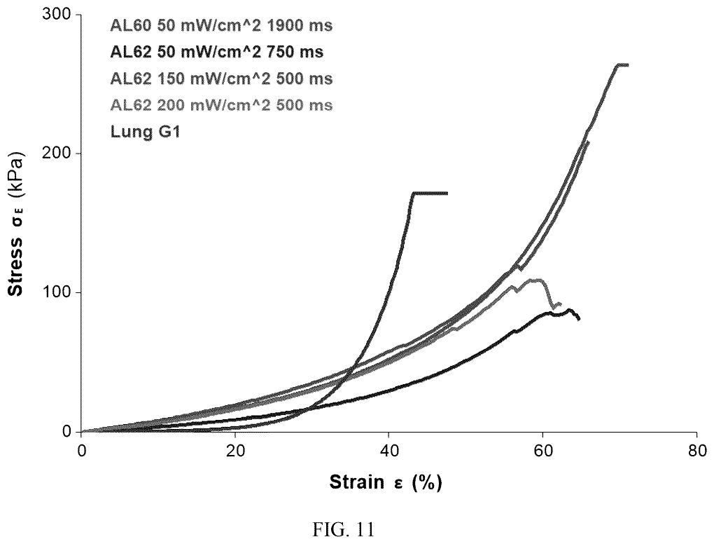

FIG. 11 illustrates a graph of Strain ε (%) vs. Stress σε (kPa) of an advanced manufactured interpenetrating polymer network (AM-IPN) for Polyethylenimine (PEI) 750,000 in synthetic AL60 (power of 50 mW/cm², time of 1900 ms), AL62 (power of 50 mW/cm², time of 750 ms), AL62 (power of 150 mW/cm², time of 500 ms), AL62 (power of 200 mW/cm², time of 500 ms) Bioinks and Lung G1.

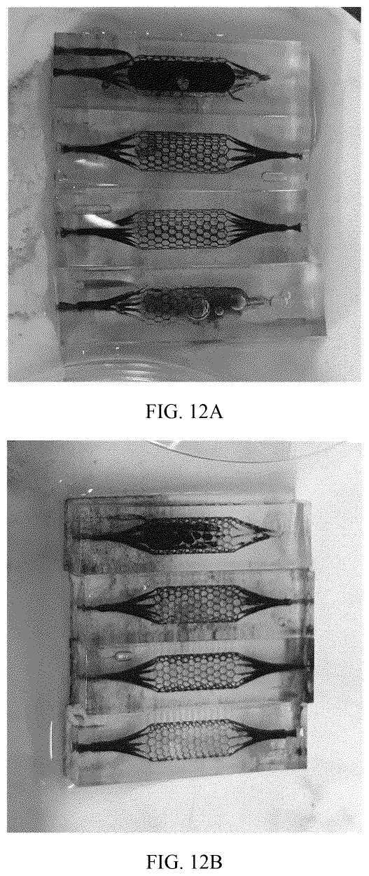

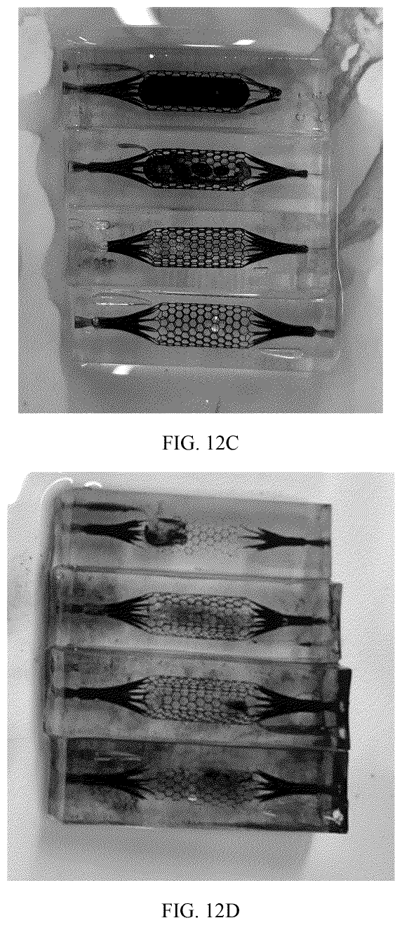

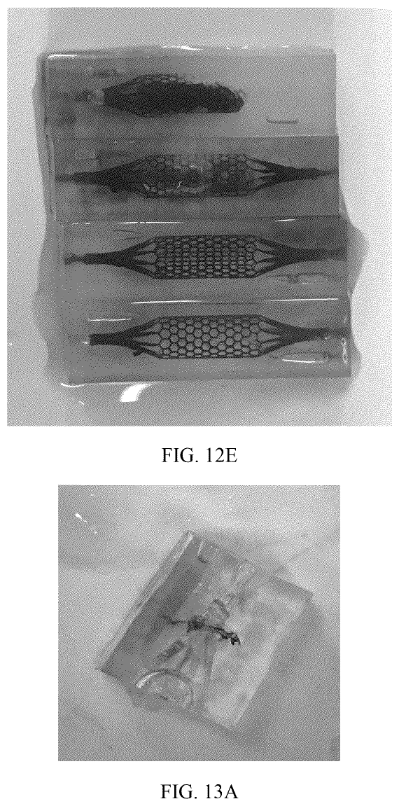

FIG. 12A illustrates a photograph of an advanced manufactured interpenetrating polymer network (AM-IPN) for Polyethylenimine (PEI) in synthetic AL60 Bioink.

FIG. 12B a photograph of an advanced manufactured interpenetrating polymer network (AM-IPN) for Polyethylenimine (PEI) in synthetic AL61 Bioink.

FIG. 12C a photograph of an advanced manufactured interpenetrating polymer network (AM-IPN) for Polyethylenimine (PEI) in synthetic AL62 (power of 50 mW/cm²) Bioink, showing a low crosslinking density of the interpenetrating polymer network of PEI in the Bioink.

FIG. 12D a photograph of an advanced manufactured interpenetrating polymer network (AM-IPN) for Polyethylenimine (PEI) in synthetic AL62 (150 mW/cm²) Bioink, showing a moderate crosslinking density of the interpenetrating polymer network of PEI in the Bioink.

FIG. 12E a photograph of an advanced manufactured interpenetrating polymer network (AM-IPN) for Polyethylenimine (PEI) in synthetic AL62 (200 mW/cm²) Bioink, showing a low crosslinking density of the interpenetrating polymer network of PEI in the Bioink.

AL60 Bioink Compression Data

| Area Under Curve | % Maximum Strain |
|---|---|
| 3796.09 | 286.60 |
| 4427.06 | 261.01 |
| 4435.10 | 286.20 |
| 2818.34 | 172.02 |
| 3811.92 | 132.85 |
| 4159.17 | 161.70 |
| 3907.94 | 216.73 |

AL62 (50 mW/cm², 2750 ms) Bioink Compression Data

| Area Under Curve | % Maximum Strain |
|---|---|
| 3224.50 | 183.94 |
| 1888.31 | 94.14 |
| 1902.74 | 85.04 |
| 2686.84 | 153.69 |
| 4899.90 | 256.91 |
| 2834.02 | 154.58 |
| 2906.05 | 154.72 |

AL62 (150 mW/cm², 500 ms) Bioink Compression Data

| Area Under Curve | % Maximum Strain |
|---|---|
| 3830.12 | 211.54 |
| 4232.07 | 233.76 |
| 5645.61 | 265.12 |
| 3123.30 | 172.02 |
| 3811.92 | 218.95 |
| 4959.65 | 263.49 |
| 4267.11 | 227.48 |

AL62 (200 mW/cm², 2500 ms) Bioink Compression Data

| Area Under Curve | % Maximum Strain |
|---|---|
| 2133.17 | 108.33 |
| 2544.82 | 134.07 |
| 2586.95 | 152.72 |
| 1834.83 | 101.74 |
| 2962.35 | 167.12 |
| 2302.73 | 109.05 |
| 2394.14 | 128.84 |

The AL62 Bioink, when printed at 50 mW/cm², resulted in a softer hydrogel than the AL60 Bioink (Stock); and the AL62 Bioink, when printed at 150 mW/cm², resulted in a tougher hydrogel than the AL60 Bioink (Stock).

The Polyethylenimine (PEI) polymer was incorporated into AL60 Bioink (Stock) when printed at 150 mW/cm² and 200 mW/cm².

Suture Testing

Using a 45 mm gauge needle, the needle was passed through a solid part of an advanced manufactured hydrogel material (capsulent). The hydrogel material (capsulent) was inspected for any cracking. The purpose of this test was to see if the hydrogel material could be sutured without cracking or falling apart.

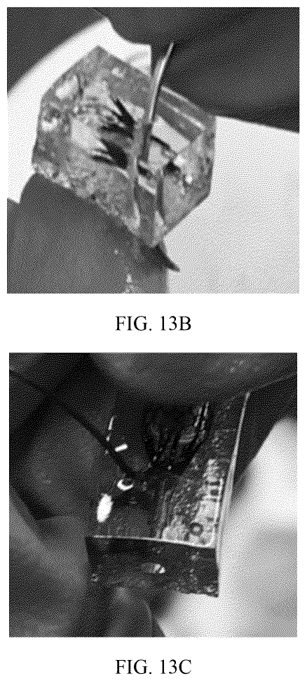

FIG. 13A illustrates a photograph of a suture test of an advanced manufactured interpenetrating polymer network (AM-IPN) for Polyethylenimine (PEI) in synthetic AL62 (power of 50 mW/cm², time of 750 ms) Bioink, showing a low crosslinking density of the interpenetrating polymer network of PEI in the Bioink.

FIG. 13B illustrates a photograph of a suture test of an advanced manufactured interpenetrating polymer network (AM-IPN) for Polyethylenimine (PEI) in synthetic AL62 (power of 100 mW/cm², time of 500 ms) Bioink, showing a low crosslinking density of the interpenetrating polymer network of PEI in the Bioink.

FIG. 13C illustrates a photograph of a suture test of an advanced manufactured interpenetrating polymer network (AM-IPN) for Polyethylenimine (PEI) in synthetic AL62 (power of 150 mW/cm², time of 450 ms) Bioink, showing moderate crosslinking density of the interpenetrating polymer network of PEI in the Bioink.

The AL62 Bioink, when printed at 50 mW/cm² and 100 mW/cm², failed the suture test. However, the AL62 Bioink, when printed at 150 mW/cm² and 200 mW/cm² passed the suture test.

Polyvinylpyrrolidone (PVP)

In these examples, a Polyvinylpyrrolidone (PVP) polymer was incorporated into AI28 and AJ55 Bioinks, as follows.

The PVP polymer has a bulky side chain so the polymer could potentially cause stiffness in AI28 Bioink.

Polyvinylpyrrolidone (PVP) 130000

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.95-1.05 |
| 130000 | | MKCP1726 | |
| Water | | | 98.95-99.05 |
| Total | | | 100 |

AI28 Bioink (Stock)

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 2.85-3.15 |
| NAP | 1.52-1.68 |
| UV386A | 0.152-0.168 |
| Water | 76.23-84.25 |
| Total | 100 |

AL67 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.95-1.05 |
| 130000 | | MKCP1726 | |
| Water | | | 46.78-51.70 |
| AI28 | | | 47.27-52.25 |
| (Stock) | | | |
| Total | | | 100 |

AL68 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.475-0.525 |
| 130000 | | MKCP1726 | |
| Water | | | 47.25-52.23 |
| AI28 | | | 47.27-52.25 |
| (Stock) | | | |
| Total | | | 100 |

AL69 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.095-0.105 |
| 130000 | | MKCP1726 | |
| Water | | | 47.63-52.65 |
| AI28 | | | 47.27-52.25 |
| (Stock) | | | |
| Total | | | 100 |

AL70 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.048-0.053 |
| 130000 | | MKCP1726 | |
| Water | | | 47.68-52.70 |
| AI28 | | | 47.27-52.25 |
| (Stock) | | | |
| Total | | | 100 |

AI19 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 4.75-5.25 |
| PEGDA 6000 | 4.75-5.25 |
| PEGDA 575 | 2.85-3.15 |
| NAP | 1.9-2.1 |
| UV386A | 0.133-0.147 |
| Water | 80.62-89.10 |
| Total | 100 |

AL59 Bioink

| | | | | |
|---|---|---|---|---|
| | Molecular | Registry | Stock | |
| Reagent | Weight (MW) | No. | Solution (%) | Amount (%) |

| | |
|---|---|
| HPA | 4.75-5.25 |
| PEGDA | 4.75-5.25 |
| 6000 | |
| NAP | 1.52-1.68 |
| UV386A | 0.114-0.126 |
| Water | 75.75-83.73 |
| Total | 100 |

AL71 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.95-1.05 |
| 130000 | | MKCP1726 | |
| Water | | | 49.76-55.0 |
| AI19 | | | 44.29-48.95 |
| (Stock) | | | |
| Total | | | 100 |

AI15 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| HPA | 130.4 | HPA | 9.5-10.5 |
| | | 97699847GO | |
| | | 10Nov20 | |
| PEGDA | 6052 | PEGDA 6000 - | 4.75-5.25 |
| 6000 | | BB0308-78 | |
| TMPTA | 956 | SR9035 - | 0.95-1.05 |
| 912 | | LDJ1138 | |
| NAP | 311.26 | NAP -- | 1.43-1.58 |
| | | 0010769 | |
| UV386A | 478.03 | UV386A - | 0.152-0.168 |
| | | 0917-20A- | |
| | | 386A | |
| Water | | | 76.32-84.36 |
| Total | | | 100 |

AJ55 Bioink (Stock)

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 1.9-2.1 |
| NAP | 0.95-1.05 |
| UV386A | 0.171-0.189 |
| Water | 76.78-84.86 |
| Total | 100 |

AI15 Bioink (Stock)

| | | | | |
|---|---|---|---|---|
| | Molecular | Registry | Stock | |
| Reagent | Weight (MW) | No. | Solution (%) | Amount (%) |

| | | | |
|---|---|---|---|
| HPA | 130.4 | HPA | 9.5-10.5 |
| | | 97699847GO | |
| | | 10Nov20 | |
| PEGDA | 6052 | PEGDA 6000 - | 4.75-5.25 |
| 6000 | | BB0308-78 | |
| TMPTA | 956 | SR9035 - | 0.95-1.05 |
| 912 | | LDJ1138 | |
| NAP | 311.26 | NAP -- | 1.425-1.575 |
| | | 0010769 | |
| UV386A | 478.03 | UV386A - | 0.152-0.168 |
| | | 0917-20A- | |
| | | 386A | |
| Water | | | 28.5-31.5 |
| Total | | | 100 |

AL72 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.95-1.05 |
| 130000 | | MKCP1726 | |
| Water | | | 47.82-52.86 |
| AI15 | | | 45.28-50.04 |
| (Stock) | | | |
| Total | | | 100 |

AL73 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.475-0.525 |
| 130000 | | MKCP1726 | |
| PEGTA | 956 | SR9035 - | 0.95-1.05 |
| 912 | | LDJ1138 | |
| Water | | | 48.30-53.38 |
| AI15 | | | 45.28-50.04 |
| (Stock) | | | |
| Total | | | 100 |

AL74 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.095-0.105 |
| 130000 | | MKCP1726 | |
| PEGTA | 956 | SR9035 - | 0.95-1.05 |
| 912 | | LDJ1138 | |
| Water | | | 48.68-53.80 |
| AI15 | | | 45.28-50.04 |
| (Stock) | | | |
| Total | | | 100 |

AL75 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVP | 130000 | PVP 130000 - | 0.048-0.053 |
| 130000 | | MKCP1726 | |
| PEGTA | 956 | SR9035 - | 0.95-1.05 |
| 912 | | LDJ1138 | |
| Water | | | 48.73-53.85 |
| AI15 | | | 45.28-50.04 |
| (Stock) | | | |
| Total | | | 100 |

Rheology Testing

The rheology testing is discussed above and shown in FIGS. 2A-2C-8.

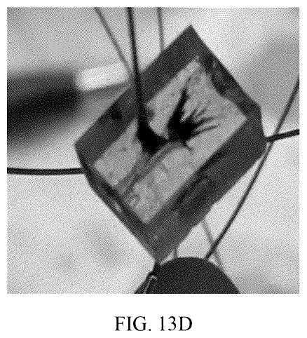

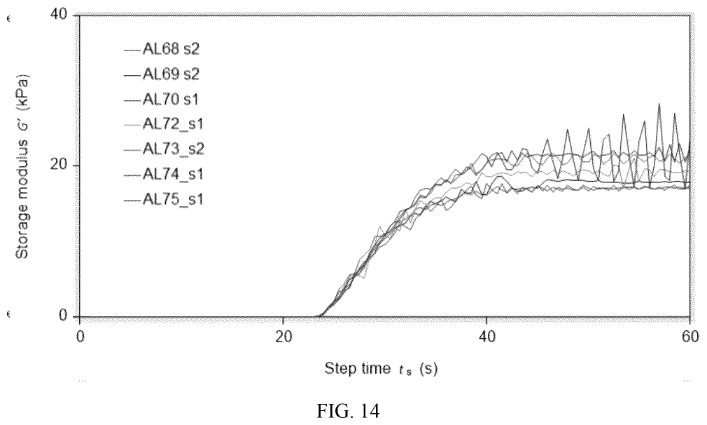

FIG. 14 illustrates a graph of Step Time ts (s) vs. Storage Modulus G′ (kPa) of an advanced manufactured interpenetrating polymer network (AM-IPN) for Polyvinylpyrrolidone (PVP) in synthetic AL68, AL69, AL70, AL72, AL73, AL74 and AL 75 Bioinks.

Rheology Data

| Bioink | Onset Time | Storage Modulus at 50 s |
|---|---|---|
| AL68 | 3 | 16.77 |
| AL69 | 3.5 | 17.97 |
| AL70 | 2 | 18.71 |
| AL72 | 3 | 17.39 |
| AL73 | 3 | 20.67 |

AL68, AL70, AL72, AL73 and AL74 Bioinks Print
Settings for FS20 DLP 3D System Printer

| | | | Region | | |
|---|---|---|---|---|---|
| Baselayer | B | Units | Layer | E | Units |
| Power | 50 | mW/cm² | Power | 50 | mW/cm² |
| Exposure | 2000 | ms | Exposure | 1700 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

Compression Testing

The compression testing is discussed above and shown in FIGS. 3A-3G-3.

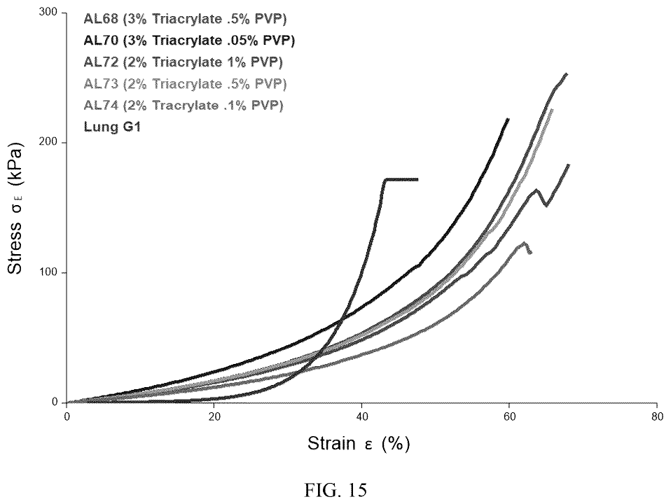

FIG. 15 illustrates a graph of Strain ε (%) vs. Stress σε (kPa) of an advanced manufactured interpenetrating polymer network (AM-IPN) for Polyvinylpyrrolidone (PVP) in synthetic AL68 (3% Triacrylate, 0.5% PVP), AL70 (3% Triacrylate, 0.05% PVP), AL72 (2% Triacrylate, 1% PVP), AL73 (2% Triacrylate, 0.5% PVP), AL74 (2% Triacrylate, 0.1% PVP) Bioinks and Lung G1.

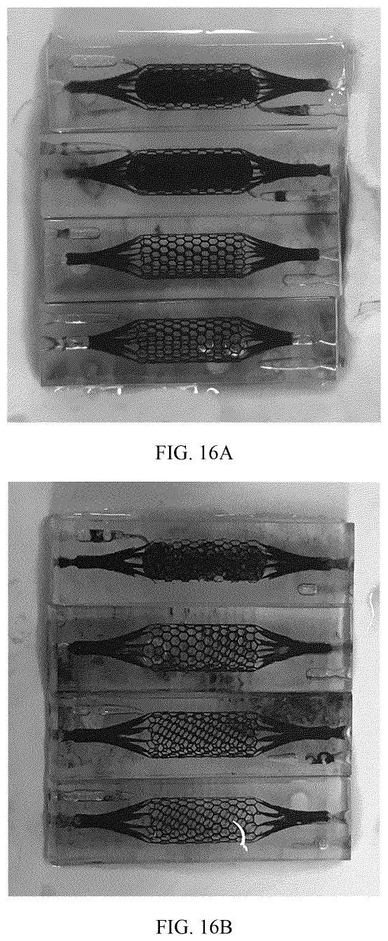

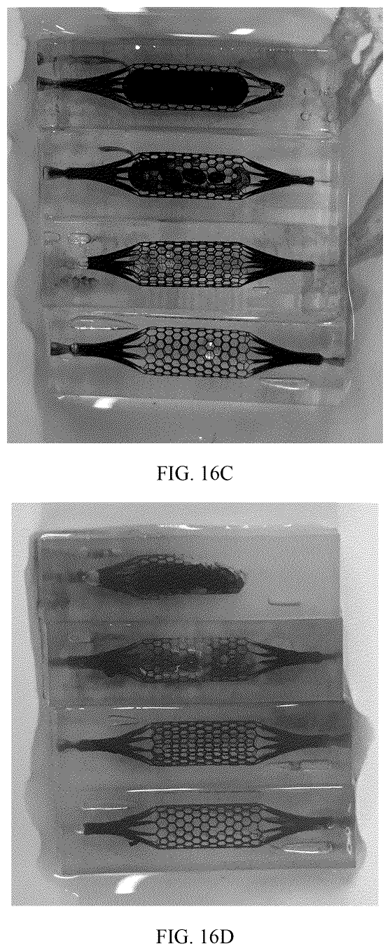

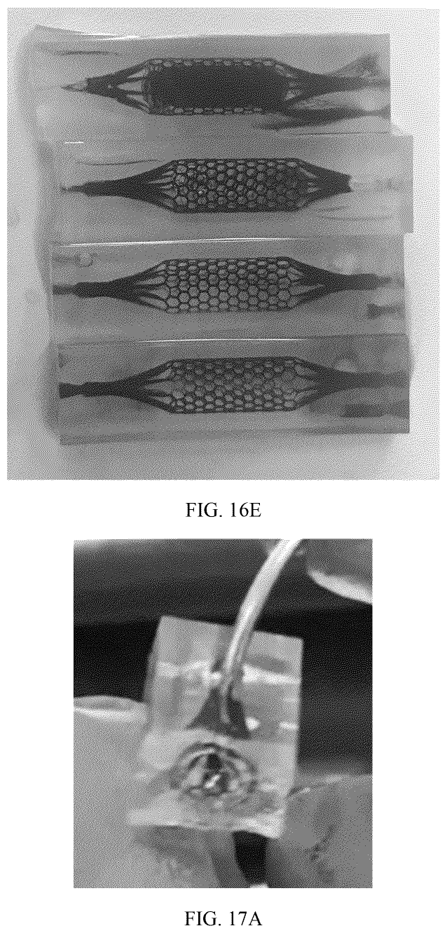

FIG. 16A illustrates a photograph of an advanced manufactured interpenetrating polymer network (AM-IPN) for Polyvinylpyrrolidone (PVP) in synthetic AL68 Bioink.

FIG. 16B a photograph of an advanced manufactured interpenetrating polymer network (AM-IPN) for Polyvinylpyrrolidone (PVP) in synthetic AL70 Bioink.

FIG. 16C a photograph of an advanced manufactured interpenetrating polymer network (AM-IPN) for Polyvinylpyrrolidone (PVP) in synthetic AL72 Bioink.

FIG. 16D a photograph of an advanced manufactured interpenetrating polymer network (AM-IPN) for Polyvinylpyrrolidone (PVP) in synthetic AL73 Bioink.

FIG. 16E a photograph of an advanced manufactured interpenetrating polymer network (AM-IPN) for Polyvinylpyrrolidone (PVP) in synthetic AL74 Bioink.

AL68 Bioink Compression Data

| Area Under Curve | % Maximum Strain |
|---|---|
| 3204.69 | 199.05 |
| 4373.49 | 251.27 |
| 3337.19 | 184.19 |
| 2879.96 | 181.19 |
| 4374.83 | 260.40 |
| 4347.76 | 263.93 |
| 3752.98 | 223.47 |

AL70 Bioink Compression Data

| Area Under Curve | % Maximum Strain |
|---|---|
| 2912.44 | 174.48 |
| 2270.52 | 124.39 |
| 4008.33 | 245.69 |
| 4311.78 | 253.34 |
| 3805.09 | 232.96 |
| 3389.07 | 196.91 |
| 3449.54 | 204.63 |

AL72 Bioink Compression Data

| Area Under Curve | % Maximum Strain |
|---|---|
| 3423.97 | 209.84 |
| 3032.70 | 155.24 |
| 3281.38 | 207.95 |
| 3695.25 | 183.57 |
| 3231.84 | 188.50 |
| 3150.41 | 176.93 |
| 3302.59 | 187.01 |

AL73 Bioink Compression Data

| Area Under Curve | % Maximum Strain |
|---|---|
| 4027.78 | 255.23 |
| 3852.70 | 234.92 |
| 4421.14 | 207.95 |
| 3695.25 | 239.71 |
| 4094.71 | 236.51 |
| 3533.24 | 212.05 |
| 3937.47 | 231.061 |

AL74 Bioink Compression Data

| Area Under Curve | % Maximum Strain |
|---|---|
| 2042.94 | 126.11 |
| 3359.99 | 196.88 |
| 2062.54 | 122.42 |
| 1988.54 | 103.10 |
| 2957.11 | 181.29 |
| 1657.71 | 86.71 |
| 2344.81 | 136.09 |

The Polyvinylpyrrolidone (PVP) polymer was incorporated into AI28 Bioink (Stock) and AJ55 Bioink (Stock).

The AL73 Bioink had the best toughness and highest maximum strain while maintaining flexibility.

Suture Testing

Using a 45 mm gauge needle, the needle was passed through a solid part of an advanced manufactured hydrogel material (capsulent). The hydrogel material (capsulent) was inspected for any cracking. The purpose of this test was to see if the hydrogel material could be sutured without cracking or falling apart.

FIG. 17A illustrates a photograph of a suture test of an advanced manufactured interpenetrating polymer network (AM-IPN) for Polyvinylpyrrolidone (PVP) in synthetic AL68 Bioink.

FIG. 17B illustrates a photograph of a suture test of an advanced manufactured interpenetrating polymer network (AM-IPN) for Polyvinylpyrrolidone (PVP) in synthetic AL70 Bioink.

FIG. 17C illustrates a photograph of a suture test of an advanced manufactured interpenetrating polymer network (AM-IPN) for Polyvinylpyrrolidone (PVP) in synthetic AL73 Bioink.

The AL70 Bioink failed the suture test. However, the AL68 and AL73 Bioinks passed the suture test.

Polyvinyl Alcohol (PVA)

In these examples, Polyvinyl Alcohol (PVA) polymer was incorporated into AI28 Bioink (Stock), as follows.

Polyvinyl Alcohol (PVA) (2% PVP Stock Solution)

Polyvinyl alcohol (PVA) was added to water, heated at 200° C., and mixed at 200 RPM before incorporating into the bioink.

AI28 Bioink (Stock)

| | | | | |
|---|---|---|---|---|
| | Molecular | Registry | Stock | |
| Reagent | Weight (MW) | No. | Solution (%) | Amount (%) |

| | |
|---|---|
| HPA | 9.5-10.5 |
| PEGDA 6000 | 4.75-5.25 |
| TMPTA 912 | 2.85-3.15 |
| NAP | 1.52-1.68 |
| UV386A | 0.152-0.168 |
| Water | 76.23-84.25 |
| Total | 100 |

AL76 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVA | 93500 | PVA 93500 - | 0.95-1.05 |
| | | MKBZ0546V | |
| Water | | | 46.78-51.70 |
| AI28 | | | 47.27-52.25 |
| (Stock) | | | |
| Total | | | 100 |

AL77 Bioink

| | | | | |
|---|---|---|---|---|
| | Molecular | Registry | Stock | |
| Reagent | Weight (MW) | No. | Solution (%) | Amount (%) |

| | | | |
|---|---|---|---|
| PVA | 93500 | PVA 93500 - | 0.475-0.525 |
| | | MKBZ0546V | |
| Water | | | 47.25-52.23 |
| AI28 | | | 47.27-52.25 |
| (Stock) | | | |
| Total | | | 100 |

Rheology Testing

The rheology testing is discussed above and shown in FIGS. 2A-2C-8.

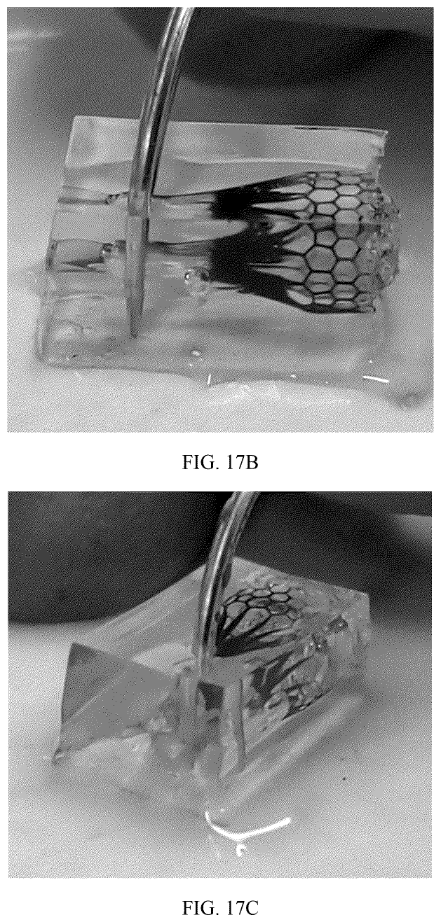

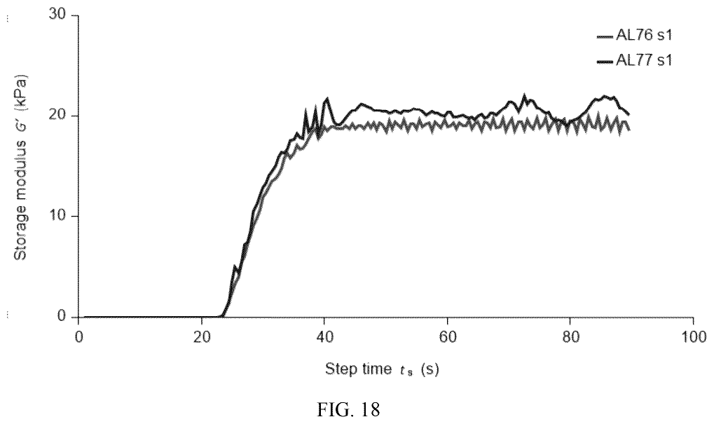

FIG. 18 illustrates a graph of Step Time ts (s) vs. Storage Modulus G′ (kPa) of advanced manufactured interpenetrating polymer network (AM-IPN) for Polyvinyl Alcohol (PVA) in synthetic AL76 and AL77 Bioinks.

Rheology Data

| Bioink | Onset Time | Storage Modulus at 50 s |
|---|---|---|
| AL76 | 23 | 19.55 |
| AL77 | 23 | 20.62 |

AL76 and AL77 Bioinks Print Settings
for FS20 DLP 3D System Printer

| | | | Region | | |
|---|---|---|---|---|---|
| Baselayer | B | Units | Layer | E | Units |
| Power | 100 | mW/cm² | Power | 100 | mW/cm² |
| Exposure | 1000 | ms | Exposure | 700 | ms |
| Time | | | Time | | |
| Pump | 300 | μm | Pump | 300 | μm |
| Distance | | | Distance | | |
| Uptime | 1500 | ms | Uptime | 1500 | ms |
| Uptime | 1500 | ms | Uptime | 1500 | ms |
| Pause | | | Pause | | |
| Downtime | 1000 | ms | Downtime | 1500 | ms |
| Downtime | 2000 | ms | Down time | 2000 | ms |
| Pause | | | Pause | | |

Compression Testing

The compression testing is discussed above and shown in FIGS. 3A-3G-3.

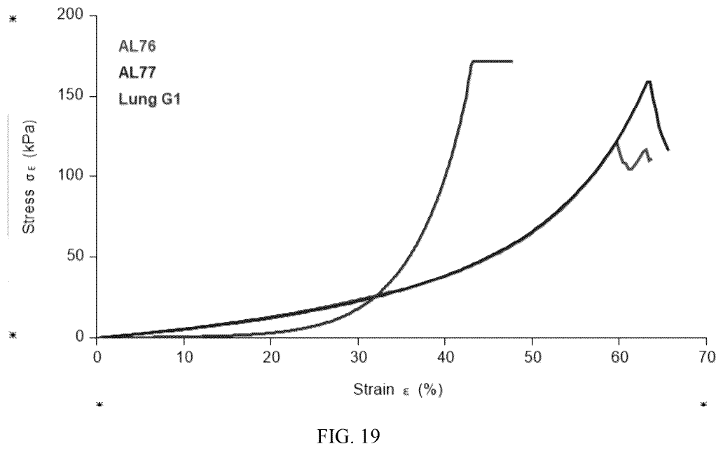

FIG. 19 illustrates a graph of Strain ε (%) vs. Stress σε (kPa) of an advanced manufactured interpenetrating polymer network (AM-IPN) for Polyvinyl Alcohol (PVA) in synthetic AL76, AL77 Bioinks and Lung G1.

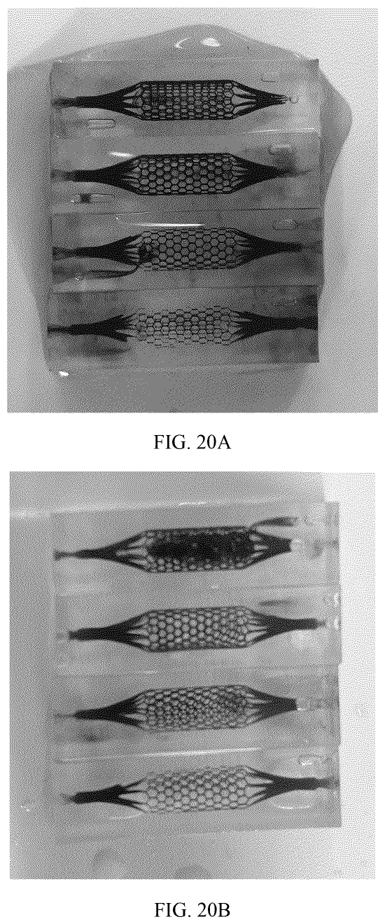

FIG. 20A illustrates a photograph of an advanced manufactured interpenetrating polymer network (AM-IPN) for Polyvinyl Alcohol (PVA) in synthetic AL76 (power of 50 mW/cm², time of 1000 ms) Bioink, showing a low crosslinking density of the interpenetrating polymer network of PVA in the Bioink. As shown in FIG. 20A, the Polyvinyl Alcohol (PVA) polymer was not incorporated into the AI28 Bioink (Stock) when printed at 50 mW/cm².

FIG. 20B illustrates a photograph of an advanced manufactured interpenetrating polymer network (AM-IPN) for Polyvinyl Alcohol (PVA) in synthetic AL76 (power of 100 mW/cm², time of 700 ms) Bioink, showing a low to moderate crosslinking density of the interpenetrating polymer network of PVA in the Bioink. As shown in FIG. 20B, the Polyvinyl Alcohol (PVA) is incorporated into AI28 Bioink (Stock) when printed at 100 mW/cm².

FIG. 20C illustrates a photograph of an advanced manufactured interpenetrating polymer network (AM-IPN) for Polyvinyl Alcohol (PVA) in synthetic AL77 (power of 100 mW/cm², time of 700 ms) Bioink, showing a moderate crosslinking density of the interpenetrating polymer network of PVA in the Bioink. As shown in FIG. 19C, the Polyvinyl Alcohol (PVA) is incorporated into AI28 Bioink (Stock) when printed at 100 mW/cm².

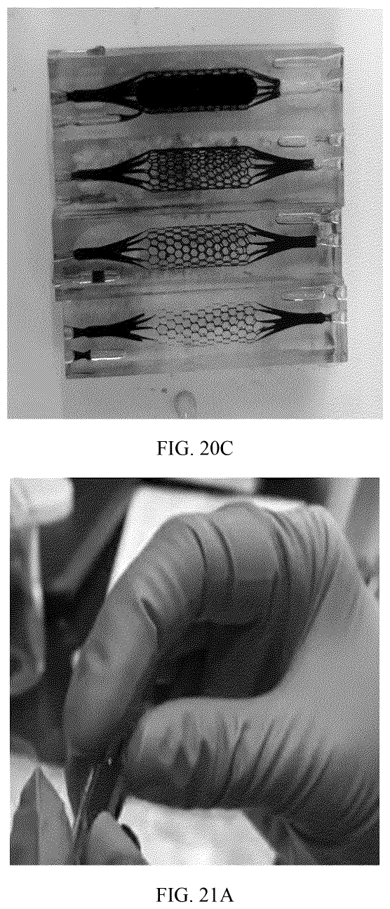

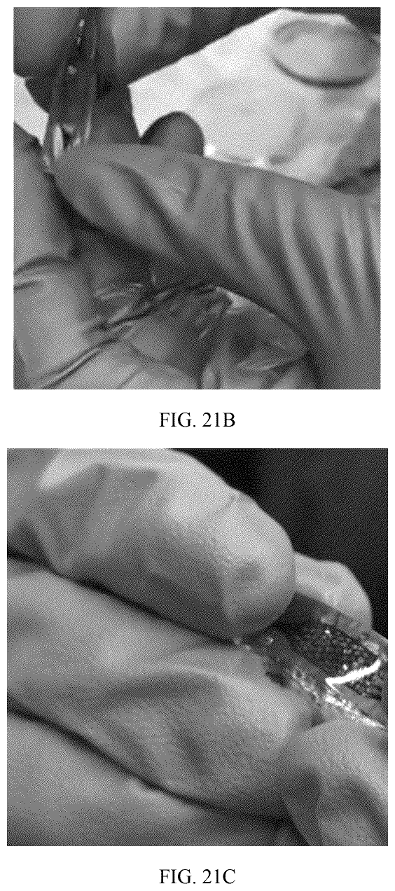

FIG. 21A illustrates a photograph of an advanced manufactured interpenetrating polymer network (AM-IPN) for Polyvinyl Alcohol (PVA) in synthetic AL76 (power of 50 mW/cm², time of 1000 ms) Bioink, showing a low crosslinking density of the interpenetrating polymer network of PVA in the Bioink. As shown in FIG. 21A, the Polyvinyl Alcohol (PVA) polymer was not incorporated into the AI28 Bioink (Stock) when printed at 50 mW/cm².

FIG. 21B illustrates a photograph of an advanced manufactured interpenetrating polymer network (AM-IPN) for Polyvinyl Alcohol (PVA) in synthetic AL76 (power of 50 mW/cm², time of 2500 ms) Bioink, showing a low to moderate crosslinking density of the interpenetrating polymer network of PVA in the Bioink. As shown in FIG. 21B, the Polyvinyl Alcohol (PVA) is incorporated into AI28 Bioink (Stock) when printed at 100 mW/cm².

FIG. 21C illustrates a photograph of an advanced manufactured interpenetrating polymer network (AM-IPN) for Polyvinyl Alcohol (PVA) in synthetic AL76 (power of 100 mW/cm², time of 700 ins) Bioink, showing a moderate crosslinking density of the interpenetrating polymer network of PVA in the Bioink. As shown in FIG. 21C, the Polyvinyl Alcohol (PVA) is incorporated into AI28 Bioink (Stock) when printed at 100 mW/cm².

AL76 Bioink Compression Data

| Area Under Curve | % Maximum Strain |
|---|---|
| 3526.93 | 248.50 |
| 1980.15 | 121.05 |
| 2283.67 | 147.30 |
| 2616.64 | 171.36 |
| 2105.07 | 129.46 |
| 2772.87 | 181.47 |
| 2547.56 | 166.52 |

AL77 Bioink Compression Data

| Area Under Curve | % Maximum Strain |
|---|---|
| 2483.12 | 159.09 |
| 2662.50 | 158.69 |
| 3775.61 | 237.50 |
| 3441.43 | 210.37 |
| 3403.70 | 222.92 |
| 2508.97 | 148.43 |
| 3046.06 | 189.50 |

The Polyvinyl Alcohol (PVA) polymer was not incorporated into AI28 Bioink (Stock) when printed at 50 m/W/cm². However, the Polyvinyl Alcohol (PVA) polymer was incorporated into AI28 Bioink (Stock) when printed at 100 mW/cm².

Suture Testing

Using a 45 mm gauge needle, the needle was passed through a solid part of an advanced manufactured hydrogel material (capsulent). The hydrogel material (capsulent) was inspected for any cracking. The purpose of this test was to see if the hydrogel material could be sutured without cracking or falling apart.

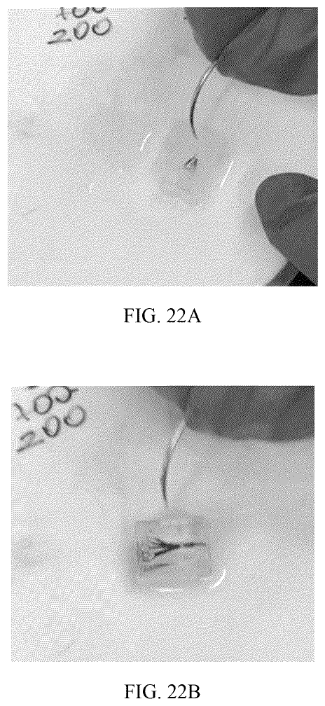

FIG. 22A illustrates a photograph of a suture test of an advanced manufactured interpenetrating polymer network (AM-IPN) for Polyvinyl Alcohol (PVA) in synthetic AL76 (power of 100 mW/cm², time of 700 ms) Bioink, showing a moderate crosslinking density of the interpenetrating network of PVA in the Bioink.

FIG. 22B illustrates a photograph of a suture test of an advanced manufactured interpenetrating polymer network (AM-IPN) for Polyvinyl Alcohol (PVA) in synthetic AL77 (power of 100 mW/cm², time of 700 ms) Bioink, showing a moderate crosslinking density of the interpenetrating polymer network of PVA in the Bioink.

The AL76 and AL77 Bioinks passed the suture test.

Interpenetrating Polymer Network Mixtures

In these examples, one or more of Polyethylene oxide (PEO), Polyvinylpyrrolidone (PVP), and Polyvinyl Alcohol (PVA) polymers were incorporated in synthetic bioinks, as follows.

Polymers

Polyethylene Oxide (PEO)

In these examples, a Polyethylene Oxide (PEO) polymer was incorporated into AW55 Bioink (Stock), as follows.

Polyethylene Oxide 4000000 (0.5% PEO Stock Solution)

Polyethylene Oxide 4000000 polymer was added to water, heated at 100° C., and mixed at 600 RPM before incorporating the bioink.

Polyethylene Oxide 400000 (0.5% PEO Stock Solution)

Polyethylene Oxide 400000 polymer was added to water, heated at 100° C., and mixed at 600 RPM before incorporating the bioink.

Polyethylene 100000 (0.5% PEO Stock Solution)

Polyethylene 100000 polymer was added to water, heated at 100° C., and mixed at 600 RPM.

Polyvinyl Alcohol (PVA)

Polyvinyl Alcohol 100000 (4% PVA Stock Solution)

Polyvinyl alcohol (PVA) was added to water, heated at 200° C., and mixed at 200 RPM before incorporating into the bioink.

Polyethylene Oxide 100000 (2% PEO Stock Solution)

Polyethylene Oxide 100000 polymer was added to water, heated at 100° C., and mixed at 600 RPM before incorporating into bioink.

Polyethylene Oxide 200000 (2% PEO Stock Solution)

Polyethylene Oxide 200000 polymer was added to water, heated at 100° C., and mixed at 600 RPM before incorporating into bioink.

Polyethylene Oxide 4000000 (0.4% PEO Stock Solution)

Polyethylene Oxide 1000000 polymer was added to water, heated at 100° C., and mixed at 600 RPM before incorporating into bioink.

AW55 Double Bioink (Stock)

| | | | | |
|---|---|---|---|---|
| | Molecular | Registry | Stock | |
| Reagent | Weight (MW) | No. | Solution (%) | Amount (%) |

| | | |
|---|---|---|
| HPA | | 19-21 |
| PEGDA | PEGDA 6000- | 9.5-10.5 |
| 6000 | BB0308-78 | |
| TAC | | 5.7-6.3 |
| NAP | | 3.04-3.36 |
| Quinoline | | 0.641-0.709 |
| Yellow | | |
| Water | | 57.12-63.13 |
| Total | | 100 |

AW77 Bioink

| | Molecular | Registry | Stock | |
|---|---|---|---|---|
| Reagent | Weight (MW) | No. | Solution (%) | Amount (%) |
| AJ55 Stock | | | 50 | 47.5-52.5 |
| Triacetate | | | | |
| PEO | 100000 | | | 0.095-0.105 |
| PEO | 400000 | | | 0.095-0.105 |
| PEO | 4000000 | | | 0.095-0.105 |
| Water | | | | 47.22-52.19 |
| Total | | | | 100 |

AW78 Bioink

| | | | | |
|---|---|---|---|---|
| | Molecular | Registry | Stock | |
| Reagent | Weight (MW) | No. | Solution (%) | Amount (%) |

| | | |
|---|---|---|
| AJ55 Stock | | 47.5-52.5 |
| Triacetate | | |
| PEO | 100000 | 0.475-0.525 |
| PEO | 400000 | 0.475-0.525 |
| PEO | 4000000 | 0.475-0.525 |
| Water | | 46.08-50.93 |
| Total | | 100 |

AW79 Bioink (4% PVA Stock Solution)

| | Molecular | Registry | Stock | |
|---|---|---|---|---|
| Reagent | Weight (MW) | No. | Solution (%) | Amount (%) |
| AJ55 Stock | | | | 47.5-52.5 |
| Triacetate | | | | 0.95-1.05 |
| PVA | 100000 | | 4 | 11.88-13.13 |
| Water | | | | 34.87-38.54 |
| Total | | | | 100 |

AW80 Bioink (4% PVA Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| Triacetate | | | | 0.95-1.05 |
| PVA | 100000 | | 4 | 23.75-26.25 |
| Water | | | | 22.8-25.2 |
| Total | | | | 100 |

AW81 Bioink (4% PVA Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| Triacetate | | | | |
| PVA | 100000 | | 4 | 47.5-52.5 |
| Water | | | | |
| Total | | | | 100 |

AW82 Bioink

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 4000000 | | | 9.5-10.5 |
| Water | | | | 38-42 |
| Total | | | | 100 |

AW83 Bioink

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 4000000 | | | 4.75-5.25 |
| Water | | | | 42.75-47.25 |
| Total | | | | 100 |

AW84 Bioink (2% PEO Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 100000 | | 2 | 9.5-10.5 |
| Water | | | | 38-42 |
| Total | | | | 100 |

AW85 Bioink (2% PEO Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 200000 | | 2 | 9.5-10.5 |
| Water | | | | |
| Total | | | | 100 |

AW86 Bioink (2% PEO Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 200000 | | 2 | 4.75-5.25 |
| Water | | | | 42.75-47.25 |
| Total | | | | 100 |

AW87 Bioink (0.4% PEO Stock Solution)

| | Molecular | | Stock | |
|---|---|---|---|---|
| Reagent | Weight (MW) | Registry No. | Solution (%) | Amount (%) |
| AJ55 Stock | | | | 47.5-52.5 |
| PEO | 4000000 | | 0.4 | 4.75-5.25 |
| Water | | | | 42.75-47.25 |
| Total | | | | 100 |

AW93 Bioink

| | Molecular | | Stock | |
|---|---|---|---|---|
| Reagent | Weight (MW) | Registry No. | Solution (%) | Amount (%) |
| AJ55 Stock | | | | 47.5-52.5 |
| PVP | Mixture | | | 1.43-1.58 |
| Water | | | | 46.08-50.93 |
| Total | | | | 100 |

AW97 Bioink

| | Molecular | | Stock | |
|---|---|---|---|---|
| Reagent | Weight (MW) | Registry No. | Solution (%) | Amount (%) |
| AJ55 Stock | | | | 47.5-52.5 |
| PVP | Mixture | | | 1.43-1.58 |
| HEMA | | | | 0.048-0.053 |
| Water | | | | 46.03-50.87 |
| Total | | | | 100 |

AW98 Bioink

| | Molecular | | Stock | |
|---|---|---|---|---|
| Reagent | Weight (MW) | Registry No. | Solution (%) | Amount (%) |
| AJ55 Stock | | | | 47.5-52.5 |
| Quinoline | | | | |
| Yellow | | | | |
| PVA | 100000 | | | 47.5-52.5 |
| Water | | | | |
| Total | | | | 100 |

AW99 Bioink

| | Molecular | | Stock | |
|---|---|---|---|---|
| Reagent | Weight (MW) | Registry No. | Solution (%) | Amount (%) |
| HPA | | | | 6.65-7.35 |
| PEGDA | | PEGDA 6000 - | | 4.75-5.25 |
| 6000 | | BB0308-88 | | |
| TAC | | | | 1.9-2.1 |
| NAP | | NAP -- | NAP -- | 1.9-2.1 |
| | | 20210208 | 20210306 | |
| Quinoline | | | 10 | 0.285-0.315 |
| Yellow | | | | |
| PVP | 1300000 | | | 0.95-1.05 |
| Water | | | | 78.57-86.84 |
| Total | | | | 100 |

AW99 Bioink (with 2/0.3% of PI/Dye) Print Settings for FS20 DLP 3D
System Printer

| | | | Region | | |
|---|---|---|---|---|---|
| Baselayer | B | Units | Layer | E | Units |
| Power | 50 | mW/cm² | Power | 100 | mW/cm² |
| | | | Layer | 1.4 | |
| | | | Time | | |
| | | | Pump | 1 | |
| | | | Every Nth | | |
| | | | Layer | | |
| Exposure Time | 125 | ms | Exposure | 125 | ms |
| | | | Time | | |
| Pump Distance | 2000 | μm | Pump | 2000 | μm |
| | | | Distance | | |
| Uptime | 1000 | ms | Uptime | 2000 | ms |
| Uptime Pause | 1000 | ms | Uptime | 1000 | ms |
| | | | Pause | | |
| Downtime | 1000 | ms | Downtime | 2000 | ms |
| Downtime Pause | 0 | ms | Down | 6000 | ms |
| | | | time | | |
| | | | Pause | | |
| Base: Glass/ | Titanium | | | Titanium | |
| Velcro/Ceramic | | | | | |
| Model Printed | RL48 | | | RL 49 | |
| Z compensation | Perfused | | | Perfused | |
| | and | | | and | |
| | ventilated | | | ventilated | |
| | for 24 | | | for 24 | |
| | hours | | | hours | |
| Future Direction | Print n = 2 | | | Print n = 2 | |

Swelling Testing

The swelling of the Bioink hydrogels (dogbones) were observed for 24 hours.

AW93 Bioink hydrogels (dogbones) swelled about 30% so IPA was decreased from 10% to 7%.

Compression Testing

The compression testing is discussed above and shown in FIGS. 3A-3G-3.

Compression failure strength and strain are the values of maximum stress (kPa) and maximum strain (%) at which the printed disc fails.

AW93 Bioink (180 mW/cm², 1 s)

| | % | | |
|---|---|---|---|
| | Maximum | Maximum | |
| Dogbone | Strain | Stress (kPa) | Modulus |
| 1 | 114.37 | 50.59 | 58.58 |
| 2 | 144.79 | 51.47 | 37.94 |
| 3 | 103.49 | 51.94 | 59.42 |
| 4 | | | |
| 5 | | | |
| 6 | | | |
| 7 | | | |
| 8 | | | |
| Average | 120.88 | 51 | 51.98 |
| Standard | 21.41 | 0.82 | 12.17 |
| Deviation | | | |

AW99 Bioink (180 mW/cm², 1 s)

| | % | | |
|---|---|---|---|
| | Maximum | Maximum | |
| Dogbone | Strain | Stress (kPa) | Modulus |
| 1 | 161.32 | 37.45 | 22.72 |
| 2 | 173.69 | 34.87 | 19.65 |
| 3 | | | |
| 4 | | | |
| 5 | | | |
| 6 | | | |
| 7 | | | |
| 8 | | | |
| Average | 167.51 | 36.16 | 21.185 |
| Standard | 8.75 | 1.82 | 2.17 |
| Deviation | | | |

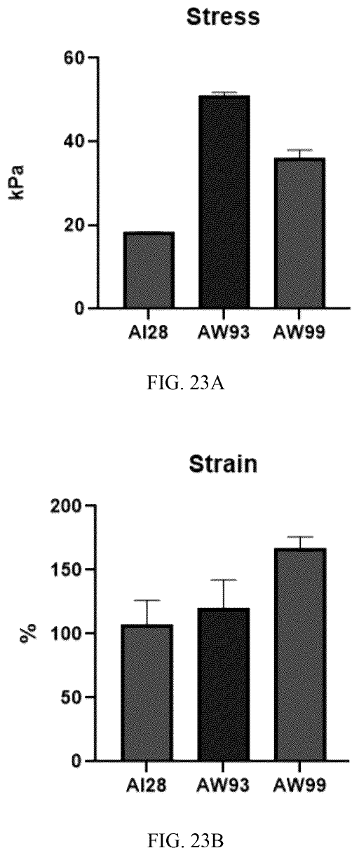

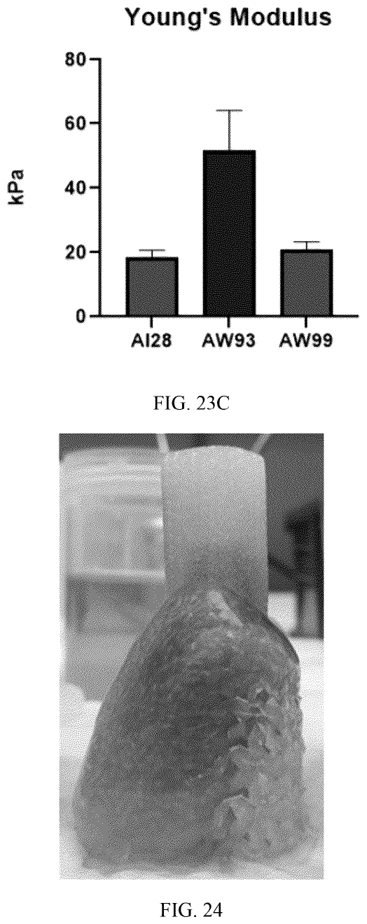

FIG. 23A illustrates a chart of Stress (kPA) for Polyvinylpyrrolidone (PVP) in synthetic AW55, AW99 and AI28 Bioinks.

FIG. 23B illustrates a chart of Strain (%) for Polyvinylpyrrolidone (PVP) in synthetic AW55, AW99 and AI28 Bioinks.

FIG. 23C illustrates a chart of Young's Modulus (kPa) for Polyvinylpyrrolidone (PVP) in synthetic AW55, AW99 and AI28 Bioinks.

FIG. 24 illustrates a photograph of a hydrogel (dogbone) material.

### Method of Making an Advanced Manufactured Interpenetrating Polymer Network (AM-IPN)

A method for making an advanced manufactured interpenetrating polymer network (AM-IPN) comprises: a) printing one or more of a primary polymer network, a secondary polymer network and one or more crosslinks using a synthetic bioink and a 3D printing technique; b) assembling and/or printing the AM-IPN as described herein to form an assembled AM-IPN.

In an embodiment, the 3D printing technique is one or more of digital light projection printing (DLP), stereolithography (SLA) printing technique, extrusion 3D printing technique or selective laser sintering 3D printing technique or a combination thereof. In an embodiment, the 3D printing technique is a digital light printing (DLP) printing technique.

### Method of Using an Advanced Manufactured Interpenetrating Polymer Network (AM-IPN)

As method of using an advanced manufactured interpenetrating network (AM-IPN) comprises a) a) modifying a surface of the AM-IPN to attach small airway epithelial cells (SAEC) to form a modified AM-IPN.

In an embodiment, the method further comprises: b) using the modified AM-IPM for perfusion and ventilation, as described herein.

The embodiments and examples set forth herein are presented to best explain the present invention and its practical application and to thereby enable those skilled in the art to make and utilize the invention. However, those skilled in the art will recognize that the foregoing description and examples have been presented for the purpose of illustration and example only. The description as set forth is not intended to be exhaustive or to limit the invention to the precise form disclosed. Many modifications and variations are possible in light of the above teaching without departing from the spirit and scope of the following claims. The invention is specifically intended to be as broad as the claims below and their equivalents.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. An advanced manufactured interpenetrating polymer network (AM-IPN) comprising:
a primary polymer network;
a secondary polymer network, wherein the secondary polymer network is bonded to the primary polymer network via one or more crosslinks,
wherein one or more of the primary polymer network, the secondary polymer network and the one or more crosslinks are printed using a synthetic bioink.

2. The AM-IPN of claim 1, wherein the synthetic bioink comprises one or more of:
HPA, in an amount from about 0.5% to about 20%;
PEGDA 6000, in an amount from about 0.5% to about 20%;
TMPTA 912, in an amount from about 0.05% to about 3%;
NAP, in an amount from about 0.05% to about 3%;
UV386A, in an amount from about 0.0001% to about 0.5%;
Polymer, in an amount from about 0.001% to about 2.0%; and
Water, in an amount as a balance.

3. The AM-IPN of claim 2, wherein the polymer comprises one or more of: polyethylene oxide (PEO), polyethylenimine (PEI), polyvinylpyrrolidone (PVP), polyvinyl alcohol (PVA), and combinations thereof.

4. The AM-IPN of claim 3, wherein the polymer is PEO having a molecular weight from about 100000 to about 4000000.

5. The AM-IPN of claim 3, wherein the polymer is PEO in an amount from about 0.005% to about 1%.

6. The AM-IPN of claim 3, wherein the polymer is PEI having a molecular weight from about 25000 to about 75000.

7. The AM-IPN of claim 3, wherein the polymer is PEI in an amount from about 0.005% to about 1%.

8. The AM-IPN of claim 3, wherein the polymer is PVP having a molecular weight from about 1000000 to about 1300000.

9. The AM-IPN of claim 3, wherein the polymer is PVP or PVA in an amount from about 0.005% to about 2%.

10. The AM-IPN of claim 3, wherein the polymer is PVA having a molecular weight from about 89000 to about 98000.

11. (canceled)

12. The interpenetrating network of claim 1, wherein the synthetic bioink comprises one or more of:
HPA, in an amount from about 0.5% to about 20%;
PEGDA 6000, in an amount from about 0.5% to about 20%;
PEGDA 3400, in an amount from about 0.2% to about 15%;
NAP, in an amount from about 0.05% to about 3%;
UV386A, in an amount from about 0.0001% to about 0.5%;
Polymer, in an amount from about 0.001% to about 2.0%; and
Water, in an amount as a balance.

13. The AM-IPN of claim 12, wherein the polymer comprises one or more of: polyethylene oxide (PEO), polyethylenimine (PEI), polyvinylpyrrolidone (PVP), polyvinyl alcohol (PVA), and combinations thereof.

14. The AM-IPN of claim 13, wherein the polymer is PEO having a molecular weight from about 100000 to about 4000000.

15. The AM-IPN of claim 13, wherein the polymer is PEO in an amount from about 0.005% to about 1%.

16. The AM-IPN of claim 13, wherein the polymer is PEI having a molecular weight from about 25000 to about 75000.

17. The AM-IPN of claim 13, wherein the polymer is PEI in an amount from about 0.005% to about 1%.

18. The AM-IPN of claim 13, wherein the polymer is PVP having a molecular weight from about 1000000 to about 1300000.

19. The AM-IPN of claim 13, wherein the polymer is PVP or PVA in an amount from about 0.005% to about 2%.

20. The AM-IPN of claim 13, wherein the polymer is PVA having a molecular weight from about 89000 to about 98000.

21. (canceled)

22. A method of making an advanced manufactured interpenetrating polymer network (AM-IPN) comprising:
printing one or more of a primary polymer network, a secondary polymer network and one or more crosslinks using a synthetic bioink and a 3D printing technique; and
assembling and/or printing the AM-IPN to form an assembled AM-IPN.

23-30. (canceled)

31. A method of using an advanced manufactured interpenetrating polymer network (AM-IPN) comprising:
modifying a surface of the AM-IPN to attach small airway epithelial cells (SAEC) to form a modified AM-IPN.

32. (canceled)
